SEQUENCE LISTING (20 sequences)

SEQ ID NO: 1 - DNA, 3673 bp, Rattus norvegicus
  codonstart (308)..(310)
  codonStop (1574)..(1576)
  polyA_signal (3615)..(3621)
gaccgaggaccgaggtgggtgacagtccgcacaatccctaggttccctaggatccctaggttttccggagttctccctgcggtccggaccctcctgaggatctcggttgtctccccatccaggacctggcgtccccaggttctctcggaggtcggggctccctctatgcttcgcgcccctcgagtccaggttccctctgtggtgcggactcctctagcccggtgcgctcagcaccctgcacccagtctcctcatacggagcctggcggggagtccggaccttgtgtccagagctgcagtggttggacatgaatgtccctcctgtcctgcttctttttcttctgtcctccgtgagagccactgagcagccgcaggtcgtcactgagcatcccagcatggatgcagccctgaccggagccaatgcctcgcatttctgggccaactacactttctctgactggcagaacttcgtgggcaggagacgctatggggctgagtcccagaaccccacagtgaaagccctgctcatcgtggcctactccttcatcattgtcttctcgctcttcggcaatgtcctggtctgtcatgtaatcttcaagaaccagcgtatgcactcggccaccagcctcttcatcgtcaacctggcagtggcagacatcatgatcacattgctcaacacgcccttcactttggtccgctttgtgaacagcacatgggtgtttgggaagggcatgtgtcatgtcagccgctttgcccagtactgttctctacatgtctcagcactgaccctgacagctatcgcagtggaccgccaccaggtcatcatgcatcccttgaagcctcggatctccatcaccaagggtgtcatatatattgctgtcatctgggtcatggctaccttcttctctctgcctcatgccatctgccagaaactgtttaccttcaagtacagtgaggacattgtgcgctccctctgcctgccggacttcccagagccagctgacctcttttggaagtacctggacctggccaccttcatcctactctacctccttccactcttcattatctcagtggcctatgctcgtgtggccaagaaattgtggctctgcaacactattggcgacgtgaccacagagcagtacctcgccctgagacgcaagaagaagaccaccgtgaagatgctggtgcttgtggtggtcctcttcgctctctgctggttccccctcaactgctatgtcctccttctgtccagcaaggccatccacaccaacaacgccctctactttgccttccactggtttgccatgagcagcacttgttataatcccttcatctactgctggctcaacgagaacttcagggtggagctgaaggcactgttgagcatgtgccagaggccgtccaagcctcaggaagacaggctgccctccccggttccttccttcagggtggcatggacggagaagagccatggtcggagggctctgctagctaatcaccaccttccctcttcccagatccagtctgggaagacagatctgtcctctgtggaacccactgtggccgtgagttaggggaagctggaagtggtgggagagggttctgtcctctcacaattgaccagactcaaggtgtcggaaagttccccagaatcagtgagatgcttgggttcctaggaacctgtccagcaccctctggtttgcaaaccttgtagaagataccatgaggtgttgtgtgtggatctttgagcaagagctccggaagccacctcagctccagcagaggctggttcagtcaacccctgtcagttgtatagcatctgccacctttgtccttccttctgctgagcacccacagcaggacccgggtcacattattggtaggcctgacgcatgtgctcagaaaagaacttcactagccagggagaacaggcatggaggccttctgaagtcattggggcaagtccaatcacacagcttcaatggccttggctctgtctatgccagacagggcccatcttggtttcttaaaatagaaattagtattgccattttgaaaaggtcaaaaagggaaagaaattaattcagccccatcgatttgttaaaaaaaaaaagcctgttaatatgctgtgaatttatctgtaactttgccttctctatgtgtgcacatttgtacttttaaagtcatgaactacatgtatccatttagattgtaataattagcaagaaatagggatatattggagtactattgaattcctccatgacaaatggggtccctcagcatatgagaagagaagagaggagaggagaggagggtaggggaggggagaggagaggagaggagaggaggggagaggaagggaagggagtggaggggagaggagaagtaagcaaggtgagggttggatcaactctaccttgtaactagtgatgggtggggcctggccctctcctaggtggttaccatcaagatgagcagtgcaccagagagccgggaggtaggctccctttctcctgacccccagaaagaagaaggggtgaatcactgttcccttttaatggacaatgtgggacttgctttccaataagctggaaataattgtgcttagacacctcatctgcccaagatgtctagacagggcatttttctccattgtctgaatgatgaaaaactttttcacaccagttccagagagaagatttccctgtctacatcacctgagaaattcatgtgtcatttttaaaaaacttacaggcttcttgaagccgctctattcctcgagggcccctttgtcagagaaatctcattccaggtgcccagtgatttcgccatcttccatgtggtcccgatgactctcagtgttccccactgacagctgtgccaggcagaactgactccaatccccatgcccagagccatggctgctgcgctagcatcggattcccacctcctcctcacagagccaccacagagacaagccatctttgtggagctgagagggaaactgaggctcaagggaggagaggctcatccaagactcatttagaactctcagttgctaactgaagcacaatctgcttaagtctgattccctctaacaaacctggggggttgcttaaactagtctgtccataggagtgttagaaaaaaatccaagataaaaaaaaatgtcatctatagaacatcagtttgtttaaaaaaaatgttttggagagggtagatttccaaatataaagtgagtttttgaggccacagcttctgcatctgtcctatctctgtattgtcagctggtacctgggagtttcatctttcatgtggccaagaagagtcatggtactaacctgaggacaggcagcagcaacacacagtgtgtcttcccctagggacttgtgggaggagcttcagccatagctcccagagggcagctggagacagcaccctgtcacagagtcagccatcagatctgcatctcttgtccgtccccatctgctcccttccttgtatgccgtcggagcagacgtgactggtaacagtgactgtggtctttgtgaagcctgggtttttccaatatgctgcaaagtgtgctttattgtgagtcaccataggcgcaataaaacctctcctctgtgtgcaaaaaaaaaaaaaaaaaaaaaaaaaaaaaaaaaaaa

SEQ ID NO: 2 - protein, 422 aa, Rattus norvegicus
MNVPPVLLLFLLSSVRATEQPQVVTEHPSMDAALTGANASHFWANYTFSDWQNFVGRRRYGAESQNPTVKALLIVAYSFIIVFSLFGNVLVCHVIFKNQRMHSATSLFIVNLAVADIMITLLNTPFTLVRFVNSTWVFGKGMCHVSRFAQYCSLHVSALTLTAIAVDRHQVIMHPLKPRISITKGVIYIAVIWVMATFFSLPHAICQKLFTFKYSEDIVRSLCLPDFPEPADLFWKYLDLATFILLYLLPLFIISVAYARVAKKLWLCNTIGDVTTEQYLALRRKKKTTVKMLVLVVVLFALCWFPLNCYVLLLSSKAIHTNNALYFAFHWFAMSSTCYNPFIYCWLNENFRVELKALLSMCQRPSKPQEDRLPSPVPSFRVAWTEKSHGRRALLANHHLPSSQIQSGKTDLSSVEPTVAVS

SEQ ID NO: 3 - DNA, 4182 bp, Homo sapiens
aaaatggtccctcacctcttgctgctctgtctcctccccttggtgcgagccaccgagccccacgagggccgggccgacgagcagagcgcggaggcggccctggccgtgcccaatgcctcgcacttcttctcttggaacaactacaccttctccgactggcagaactttgtgggcaggaggcgctacggcgctgagtcccagaaccccacggtgaaagccctgctcattgtggcttactccttcatcattgtcttctcactctttggcaacgtcctggtctgtcatgtcatcttcaagaaccagcgaatgcactcggccaccagcctcttcatcgtcaacctggcagttgccgacataatgatcacgctgctcaacacccccttcactttggttcgctttgtgaacagcacatggatatttgggaagggcatgtgccatgtcagccgctttgctcagtactgctcactgcacgtctcagcactgacactgacagccattgcggtggatcgccaccaggtcatcatgcaccccttgaaaccccggatctcaatcacaaagggtgtcatctacatcgctgtcatctggaccatggctacgttcttttcactcccacatgctatctgccagaaattatttaccttcaaatacagtgaggacattgtgcgctccctctgcctgccagacttccctgagccagctgacctcttctggaagtacctggacttggccaccttcatcctgctctacatcctgcccctcctcatcatctctgtggcctacgctcgtgtggccaagaaactgtggctgtgtaatatgattggcgatgtgaccacagagcagtactttgccctgcggcgcaaaaagaagaagaccatcaagatgttgatgctggtggtagtcctctttgccctctgctggttccccctcaactgctacgtcctcctcctgtccagcaaggtcatccgcaccaacaatgccctctactttgccttccactggtttgccatgagcagcacctgctataaccccttcatatactgctggctgaacgagaacttcaggattgagctaaaggcatcactgagcatgtgtcaaagacctcccaagcctcaggaggacaggcaaccctccccagttccttccttcagggtggcctggacagagaagaatgatggccggagggctctccttgccaataacctcctgcccacctcccaactccagtctgggaaggcagacctgtcatctgtggaacccattgtgacgatgagttagaagaggttgggaagagggagtgggaggggtctgtctccacctgaggcagggaaagagagcctattctcacacatgatcttcagagcgctggaaacacactcctgcagaagctgtaggactcttgaattcctaggaaactgtccagcctcctagccccatgtgatgtgaaaactaaaaggcaccaccaactagacatgtgttcataaattcccatctaagaaacactgggaggcacagcagcctgtatctctgaggaagaggagcgaggacaacgttggcccagatgggggctgaatcattcaactgcctccatctgtagggcagctgcttgccttacagcccttcctactgctgagcatcccgaagggagacctaaatcatactttctgggtgtggtgacccagatgcacagagctctgcttgaaacaggtacacgggccagggaaatgccagcaagccagagcgggcgtggagatttttatgcctcactttctggagtcactgggccatgatgaatcataagtcttcagtggcctagcaatatccagataagaaaggaccaacttgggttccttaaaacaaagggaaattattattgccacttagaaaaattcagaaaagcacacactcacatacacacacacaaaatcactctcttatcccatccatttgtgataacatctgtgaacatgctgtggctctatttgcaacattttccttcgtgtgtgtgattgtgtgcatgtgtgcatgacctttttttttcttttttttttttttgagatggaatctcgctctgtcacccaggttggagttcagtggcacaatctcggctcactgcaagctctgcctcccaggttcatgccgttctcctgcctcagcctccctagtagctgggactacaggtgcccgccaccatgcccggctaatcttttgcatttttagtagagacggggtttcaccgtgttagccaggatggtctcgatctcctgacctcgtgatccacctgcctaggcctcccaaagtgttgggattacaggcgtgagccaccccgcccggcccttttttttcatttatactttttcatttagattgtaatgattaacagaaacaggggtacctttcccaggggtgtccaccagaagagttgaagaagaggtgagaagatcccaaggtgaagcctgtcaccacgcagacagaactgccccctattgtagacagtggtgggaagggcctggctcacatgcccctccttagaggcagcctcaagatgaaccagggaatcaaactcaaccaggaggcatgattccttctctcctgcaattgcacaaaaacaagtggtggtgaaacattgttctgttttcacaagagatgagggagcaggctttccaatatgctggaagtaattgtgcttatacacctcatcctcccaagctgtctagaaggccatttcgctgtgttgtctaaataatgagggttttttcacaccgtttctcaaaagaagattcgctgtcaacatcattttggtggttcaggtgtcactttcacttacagaaacttatgcatccttaagagatctcaattcttaagggtctaacttgccagagaaatctcattccagggactgattgtttttatcaccttctgtctggtcctgagtgactcactgtgtcacccagaaaagagacctgtcaggcagaatcgagcttaattcacctgctctaaacaattgttactgacacactgcttttaggttttcttctagcaccagataaactctgaaatcaaagcatggatatgagctcagtaggaaactaaggtttgagggagaaagagtttgtcacttagacctcttggctgttaactgaaactgagcttgctcacatcatctgcttccctgtaatgaactatgagggttctttatcctagtttctatttatgaagatggttgaagattttcaagacacaagattttgaaaactcactgaattatgggcaaaaccatgactttcattgggcagacagagtcttcttttaagccacagggatccccagatggatttttcaggacaaagggtcatggcctgtacttattgtctctgtggtggccaagaagaagcaatggtttcatctgaaagctgccatagaaattctgtgacccagaatgagacaaggcagcaggcatacagaaggtttgtcccgctctgggaagcatggagaatgttgtacttcactcctctctgagggtgtctaggaatacagaagctccttgacttacatctcaataaacccatcgtaagtccaaaatattggaagttgaaaatgcattgaatacacccaacctactgaacatcgtagcttagccaagcctaccttaaatgctctgagaacacttatattagcctacagctgggaaaaatcatttaacacaaagcctatcttctaatgaagtatggaatatctcatgtaatttattgaatactgtactgaaggtgaaaaccagaatggttgtatggatatgcgaagtatggtttctactgaatgtgtattgctttcccatcatcataaaatttttaaaaatcctaagttgaatcattgttagtccaggaccttcagtactgccaagtcccagagaaaacccttttactctggggagaacccactgatggctcattagatctgtcccttccccgaggtccgtgtgtgactttgagcaaaaagtgacttctttttgtgaatgtaaaatttgtgaaacttgagtttttccaatgtgctgtaaagtgttttattgtaaattgctcatctcaataaaatttacttcaggtttaaaaaaaaaaaaaaaaaagtactagtcgacgcgtggccaatcgaattcccgcggccgccatggcggcgggagcatgcgacgtc

SEQ ID NO: 4 - protein, 423 aa, Homo sapiens
MVPHLLLLCLLPLVRATEPHEGRADEQSAEAALAVPNASHFFSWNNYTFSDWQNFVGRRRYGAESQNPTVKALLIVAYSFIIVFSLFGNVLVCHVIFKNQRMHSATSLFIVNLAVADIMITLLNTPFTLVRFVNSTWIFGKGMCHVSRFAQYCSLHVSALTLTAIAVDRHQVIMHPLKPRISITKGVIYIAVIWTMATFFSLPHAICQKLFTFKYSEDIVRSLCLPDFPEPADLFWKYLDLATFILLYILPLLIISVAYARVAKKLWLCNMIGDVTTEQYFALRRKKKKTIKMLMLVVVLFALCWFPLNCYVLLLSSKVIRTNNALYFAFHWFAMSSTCYNPFIYCWLNENFRIELKASLSMCQRPPKPQEDRQPSPVPSFRVAWTEKNDGRRALLANNLLPTSQLQSGKADLSSVEPIVTMS

SEQ ID NO: 5 - DNA, 4288 bp, Homo sapiens
caagagaaacaggctcggaaggcagatttcaactaacttataaaggccccctccaggaattctaggacattgaaatgaatcactcacccgggccccccaacatccaggctgcccttctacctttccacacggaggacttttttcccccctaaaaaaagcaaaggaagtttcctctctcttttgttagtgatgaccaccaggacatagaactagagcaccaagccacatggatattcctaaaaaggctgaagaggactggggtgcattccatatcaaataaatacttgttctatgagtggctgaagggatgttagttcaggctttaccactcatatgctcaagatccatcatttcttttctttatgccttagttttccctttaaaatggggaagtagcagaatgggaaattctaggatttcctcaaggatgagttagttttcaacctggaaacatgagcatttcattcatttattcaacaaatatgtattgggtctctactgtgggctaggaattgcacaagattaagagaacacagagaaattcaggtgtattatttacctctatgcaattcccagcctagtgggggaggtagaaatgtaatcagataattaggctacagcatcaaaagtgccatattagatggatgtaccaggagcccagtggaaggagaggccagtgcttcttgggatgaagcctttccacactaatatctcctttctgttacctctctccccctccccacttcctgcccccagtgaggacattgtgcgctccctctgcctgccagacttccctgagccagctgacctcttctggaagtacctggacttggccaccttcatcctgctctacatcctgcccctcctcatcatctctgtggcctacgctcgtgtggccaagaaactgtggctgtgtaatatgattggcgatgtgaccacagagcagtactttgccctgcggcgcaaaaagaagaagaccatcaagatgttgatgctggtggtagtcctctttgccctctgctggttccccctcaactgctacgtcctcctcctgtccagcaaggtcatccgcaccaacaatgccctctactttgccttccactggtttgccatgagcagcacctgctataaccccttcatatactgctggctgaacgagaacttcaggattgagctaaaggcattactgagcatgtgtcaaagacctcccaagcctcaggaggacaggccaccctccccagttccttccttcagggtggcctggacagagaagaatgatggccagagggctccccttgccaataacctcctgcccacctcccaactccagtctgggaagacagacctgtcatctgtggaacccattgtgacgatgagttagaagaggttgggaagagggagtgggaggggtctgtctccacctgaggcagggaaagagagcctattctcacacatgatcttcagagcgctggaaacacactcctgcagaagctgtaggactcttgaattcctaggaaactgtccagcctcctagccccatgtgatgtgaaaactaaaaggcaccaccaactagacatgtgttcataaattcccatctaagaaacactgggaggcacagcagcctgtatctctgaggaagaggagcgaggacaacgttggcccagatgggggctgaatcattcaactgcctccatctgtagggcagctgcttgccttacagcccttcctactgctgagcatcccgaagggagacctaaatcatactttctgggtgtggtgacccagatgcacagagctctgcttgaaacaggtacacgggccagggaaatgccagcaagccagagcgggcgtggagatttttatgcctcactttctggagtcactgggccatgatgaatcataagtcttcagtggcctagcaatatccagataagaaaggaccaacttgggttccttaaaacaaagggaaattattattgccacttagaaaaattcagaaaagcacacactcacatacacacacacaaaatcactctcttatcccatccatttgtgataacatctgtgaacatgctgtggctctatttgcaacattttccttcgtgtgtgtgattgtgtgcatgtgtgcatgacctttttttttcttttttttttttttgagatggaatctcgctctgtcacccaggttggagttcagtggcacaatctcggctcactgcaagctctgcctcccaggttcatgccgttctcctgcctcagcctccctagtagctgggactacaggtgcccgccaccatgcccggctaatcttttgcatttttagtagagacggggtttcaccgtgttagccaggatggtctcgatctcctgacctcgtgatccacctgcctaggcctcccaaagtgttgggattacaggcgtgagccaccccgcccggcccttttttttcatttatactttttcatttagattgtaatgattaacagaaacaggggtacctttcccaggggtgtccaccagaagagttgaagaagaggtgagaagatcccaaggtgaagcctgtcaccacgcagacagaactgccccctattgtagacagtggtgggaagggcctggctcacatgcccctccttagaggcagcctcaagatgaaccagggaatcaaactcaaccaggaggcatgattccttctctcctgcaattgcacaaaaacaagtggtggtgaaacattgttctgttttcacaagagatgagggagcaggctttccaatatgctggaagtaattgtgcttatacacctcatcctcccaagctgtctagaaggccatttcgctgtgttgtctaaataatgagggttttttcacaccgtttctcaaaagaagattcgctgtcaacatcattttggtggttcaggtgtcactttcacttacagaaacttatgcatccttaagagatctcaattcttaagggtctaacttgccagagaaatctcattccagggactgattgtttttatcaccttctgtctggtcctgagtgactcactgtgtcacccagaaaagagacctgtcaggcagaatcgagcttaattcacctgctctaaacaattgttactgacacactgcttttaggttttcttctagcaccagataaactctgaaatcaaagcatggatatgagctcagtaggaaactaaggtttgagggagaaagagtttgtcacttagacctcttggctgttaactgaaactgagcttgctcacatcatctgcttccctgtaatgaactatgagggttctttatcctagtttctatttatgaagatggttgaagattttcaagacacaagattttgaaaactcactgaattatgggcaaaaccatgactttcattgggcagacagagtcttcttttaagccacagggatccccagatggatttttcaggacaaagggtcatggcctgtacttattgtctctgtggtggccaagaagaagcaatggtttcatctgaaagctgccatagaaattctgtgacccagaatgagacaaggcagcaggcatacagaaggtttgtcccgctctgggaagcatggagaatgttgtacttcactcctctctgagggtgtctaggaatacagaagctccttgacttacatctcaataaacccatcgtaagtccaaaatattggaagttgaaaatgcattgaatacacccaacctactgaacatcgtagcttagccaagcctaccttaaatgctctgagaacacttatattagcctacagctgggaaaaatcatttaacacaaagcctatcttctaatgaagtatggaatatctcatgtaatttattgaatactgtactgaaggtgaaaaccagaatggttgtatggatatgcgaagtatggtttctactgaatgtgtattgctttcccatcatcataaaatttttaaaaatcctaagttgaatcattgttagtccaggaccttcagtactgccaagtcccagagaaaacccttttactctggggagaacccactgatggctcattagatctgtcccttccccgaggtccgtgtgtgactttgagcaaaaagtgacttctttttgtgaatgtaaaatttgtgaaacttgagtttttccaatgtgctgtaaagtgttttattgtaaattgctcatctcaataaaatttacttcaggtttaaaaaaaaaaaaaaaaaagtactagtcgacgcgtggccaatcgaattcccgcggccgccatggcggcgggagcatgcgacgtc

SEQ ID NO: 6 - protein, 246 aa, Homo sapiens [sequence omitted]

SEQ ID NO: 7 - DNA, 20 bp, Rattus norvegicus
agcagaggctggttcagtca

SEQ ID NO: 8 - DNA, 20 bp, Rattus norvegicus
gtacctatctcggttccagt

SEQ ID NO: 9 - DNA, 21 bp, Rattus norvegicus
ctcttcggtaatgtcctggtc

SEQ ID NO: 10 - DNA, 33 bp, Rattus norvegicus
ttataacaagtgctgctcatggcaaaccagtgg

SEQ ID NO: 11 - DNA, 26 bp, Rattus norvegicus
caaagtagagggcgttgttggtgtgg

SEQ ID NO: 12 - DNA, 25 bp, Rattus norvegicus
cctcttcgctctctgctggttcccc

SEQ ID NO: 13 - DNA, 28 bp, Rattus norvegicus
caactgctatgtcctccttctgtccagc

SEQ ID NO: 14 - DNA, 19 bp, Rattus norvegicus
cggccacagtgggttccac

SEQ ID NO: 15 - DNA, 20 bp, Rattus norvegicus
gaaccctaaggccaaccgtg

SEQ ID NO: 16 - DNA, 20 bp, Rattus norvegicus
tggcatagaggtctttacgg

SEQ ID NO: 17 - DNA, 20 bp, Rattus norvegicus
cgcacgatttccctctcagc

SEQ ID NO: 18 - DNA, 20 bp, Rattus norvegicus
tcgctctctgctggttcccc

SEQ ID NO: 19 - DNA, 40 bp, Rattus norvegicus
tcgctctctgctggttcccctcccttcatctactgctggc

SEQ ID NO: 20 - DNA, 19 bp, Rattus norvegicus
cggccacagtgggttccac

(19) United States
(12) Patent Application Publication
(10) Pub. No.: US 2005/0009023 A1
(43) Pub. Date: Jan. 13, 2005

(54) Glucocorticoid-induced receptor and methods of use

(76) Inventors: Neil Richtand, Cincinnati, OH (US); Floyd R. Sallee, Cincinnati, OH (US); Danzhao Wang, Norcross (UA)

Correspondence Address: FROST BROWN TODD, LLC, 2200 PNC CENTER, 201 E. FIFTH STREET, CINCINNATI, OH, 45202, US

(21) Appl. No.: 10/482,503

(22) PCT Filed: Jul. 3, 2002
(86) PCT No.: PCT/US02/21149

Related U.S. Application Data
(60) Provisional application No. 60/302,973, filed on Jul. 3, 2001.

(51) Int. Cl. C12Q 1/68

(52) U.S. Cl. 435/6

(57) ABSTRACT

Behavioral sensitization to phsychostimulants involves neuroadaptation of stress-responsive systems. The present invention provides glucocorticoid-induced receptor nucleotides and proteins belonging to G protein-coupled receptor superfamily. The present invention also provides methods methods for determining suscepibility of psychogenic disorders such as depression, anxiety and addiction as well as the development of treatments for central nervous system disorders through glucocorticoid-induced receptor expression modulation.

### CROSS-REFERENCE TO RELATED APPLICATIONS

This application claims the benefit of U.S. Provisional Patent Application Ser. No. 60/302,973 filed Jul. 3, 2001, which application is hereby incorporated by reference in its entirety.

### STATEMENT REGARDING FEDERALLY SPONSORED RESEARCH

This invention was made in part with Government support under Grant Nos. 60752 and 42662, awarded by the National Institutes of Health and Grant No. FD-R-001701, awarded by the Food and Drug Administration. The Government may have certain rights in this invention.

### BACKGROUND OF THE INVENTION

The present invention relates to compositions and methods of use of cell receptor nucleic acid and amino acid sequences. More particularly, the present invention relates to glucocorticoid-induced receptor genes and proteins and their use for diagnosis and treatment of central nervous system disorders.

Behavioral sensitization is a progressive and enduring enhancement of certain behaviors following repetitive stress or stimulant drug administration. Following repeated, intermittent treatment with stress or stimulant drugs, sensitized behaviors may occur more intensely with a shorter latency or at a lower dose than prior to behavioral sensitization. The ventral tegmental area, prefrontal cortex and amygdala seem to mediate the initial development of sensitization to neural substrates; whereas the nucleus accumbens and more distal structures may be involved in its expression. Sensitization is a long-lasting behavioral change accompanied by neuroadaptation, which results in new protein synthesis depending upon sustained alteration of gene expression. For example, ΔFosB accumulates in the nucleus accumbens long after chronic exposure to drugs of abuse including amphetamine, cocaine, and opiates, and NAC-1 mRNA expression remains elevated in the nucleus accumbens for 3 weeks following acute and repeated cocaine administration. These examples of neuroadaptation are relevant to the human addictive process in which learned drug-seeking and other behaviors persist long after bouts of drug use are extinguished.

Mechanisms of cross-sensitization between stress and psychostimulants remain elusive, but activation of the hypothalamic-pituitary-adrenal axis in response to stress may facilitate the development of long-term behavioral change and enhance the vulnerability to drug addiction. Substance P appears to be a pivotal neurotransmitter implicated in both stress and drug dependence models. The glucocorticoid-induced receptor (GIR) was originally identified as a stress-response element from a murine thymoma cDNA library treated with glucocorticoid and forskolin. GIR mRNA was further located to limbic forebrain regions, dorsal and ventral striatum, and hypothalamic nuclei within the mouse central nervous system. Recently, the gene structure of human GIR cDNA was described. Though it is known that GIR expression is restricted to brain and thymus expression and can be induced by dexamethasone and forskolin, its ligand and function remain unknown.

It has now been found that the human glucocorticoid-induced receptor plays an important role in the long-term maintenance of neuroadaptation at the genetic transcriptional level.

### BRIEF SUMMARY OF THE INVENTION

The present invention concerns methods for diagnosing and detecting a genetic potential susceptibility to addiction or stress-related disorders, including but not limited to, alcoholism, obesity, Tourette Syndrome, attention deficit disorder and post-traumatic stress disorders and the like, smoking, substance abuse and drug addiction, in a human. The methods comprise initially obtaining a DNA sample of an individual and then determining the presence or absence of particular glucocorticoid-induced receptor gene alleles in the sample. Detection of these alleles in the sample is indicative of predilection to addiction or stress-related disorders. Preferably the methods detect a predisposition to impulsive addictive and compulsive disorders such as, but not limited to, alcoholism, obesity, Tourette Syndrome, attention deficit disorder and post-traumatic stress disorders and the like, smoking, substance abuse and drug addiction, and major depression, PTSD, panic disorder, Bipolar affective disorder, borderline personality, and schizophrenic disorder.

The present invention relates to glucocorticoid-induced receptor sequences. This invention also relates to genomic DNA fragments containing regulatory and coding sequences for the glucocorticoid-induced receptor and transgenic animals made using these fragments or mutated fragments. The genomic clones demonstrate the role of the glucocorticoid-induced receptor in the central nervous system and in the pharmacological response to neuroadaptation of stress responsive systems. The invention also relates to vectors containing the DNA sequences, cells transformed with the vectors, transgenic animals carrying the sequences, cell lines derived from these transgenic animals and kits for detection of polymorphisms, and expression of protein to screen for therapeutic drugs. In addition, the invention describes the uses of all of the above.

The present invention relates to molecular genetic evidence that alleles in the human glucocorticoid-induced receptor gene are more significantly associated with the stress reward disorders than with controls. The occurrence of these disorder associated polymorphisms has a significant predictive value in the classification of subtypes of stress reward disorders.

The tendency of certain individuals to display compulsive disorder behavior is well known and includes individuals with an excessive desire for substances classes as psychoactive drugs including, but not limited to, alcohol, opiates and food. The cause and effect of substance abuse appears to be biogenic and the ability to identify gene segments associated with specific substance abuse behavior will allow development of predictive tests for compulsive disorder behavior patterns.

Restriction Fragment Length Polymorphism (RFLP) offers a powerful molecular genetic tool for the direct analysis of the human genome to determine elements that provide predisposition to genetic disease. This technique may be used to demonstrate a structural mutation in the gene that codes for a protein involved in neuroadaptation. Access to sequence variation in the human genome allows construction of genetic linkage maps through the techniques of RFLPs. This technique provides probes that are isolated from chromosome specific libraries constructed to contain some portion of human DNA.

In the present invention, the DNA probe for glucocorticoid-induced receptor gene permits evaluation of polymorphisms on the gene in the region close to the gene which could modify the function of the gene as a valuable predictor of central nervous system disorders and addiction and neural adaptation disorders. This permits use as a valuable predictor of substance abuse.

In a preferred embodiment, the present invention comprises the detection of a genetic potential susceptibility to a compulsive disease such as alcoholism or addiction to psychostimulants.

The present invention also provides for a kit for use in genetically detecting potential susceptibility to memory stress and reward disorders.

Generally, the kit comprises a restriction enzyme capable of cleaving a human glucocorticoid-induced receptor gene, a hybridization probe for detecting a human glucocorticoid-induced receptor gene allele whose presence indicates susceptibility to memory stress or reward disorders. A further kit would include PASA primers and the ingredients for PCR amplification of specific gene sequences. The ingredients for PCR amplification include AMPLITAQ® for the detection of the allele.

The alleles described herein could be detected by other methods, for example, the RFLPs detected by southern hybridization technology or other sequences specific methodologies and specific antibodies.

The above-described methods may also be of value in genetically detecting vulnerability toward other substance abuse patterns including, but not limited to, nicotine, narcotics, and other abused drugs. The methods may also be used to detect a vulnerability to attention deficit disorder and attention deficit disorder with hyperactivity.

In addition, the present invention provides methods for detection and diagnosis of long-term neuro-adaptation of stress and psychostimulant activity and the detection and diagnosis of sensitization and enhanced vulnerability to drug addiction.

Memory disorders, along with stress and addiction disorders, are likely the result of multiple factors and is polygenic in nature of which the glucocorticoid-induced receptor gene is one key factor.

### BRIEF DESCRIPTION OF THE DRAWINGS

FIG. 1 shows the 3.6 kb nucleotide sequence and deduced amino acid sequences of rat glucocorticoid-induced receptor gene (Genbank Accession Number AY029071). The amino acid sequence encoded by the longest open reading frame is shown using the single letter amino acid code. The translation start codon and stop codon are in bold. The polyadenylation signal is boxed.

FIG. 2 shows a hydropathy analysis. Plot of hydrophobicity and hydrophilicity of the rat GIR protein. Eight hydrophobic domains including a putative signal sequences (S) and seven transmembrane spans (I-VII) are predicted.

FIG. 3 shows the amino acid sequence of rat GIR and its comparison with other receptors:
A. The amino acid sequences of rat GIR, mouse GIR, human GIR, rat substance P-receptor (NK1R), rat neurokinin B receptor (NK3R), and rat neurokinin A receptor (NK2R) are aligned for best homology. Transmembrane domains (TM) of the rat GIR are predicted and labeled. Signal peptide is predicted and boxed. ▪ indicates Cys residues, ▴ indicates potential glycosylation sites, ● indicates potential protein kinase C phosphorylation sites, ◯ indicates potential protein kinase A phosphorylation sites. * indicates the aspartic acid residue. \hat{ } indicates His residues.
B. Relatedness of GIR to other members of the 7 transmembrane receptor family. A dendrogram was constructed by Pileup (GCG program) of the amino acid sequences among the cloned members of the 7 transmembrane family receptors (TSHR, Thyrotropin receptor; LHR, Lutropin receptor; NY4R, neuropeptide Y4 receptor; NY1R, neuropeptide Y1 receptor; GIR, glucocorticoid receptor, NK1R, substance P-receptor; NK3R, neurokinin B receptor; NK2R, neurokinin A receptor; OPRK, kapp-type opioid receptor; D3R, Dopamine D3 receptor; D2R, Dopamine D2 receptor; D5R, Dopamine D5 receptor; D1R, Dopamine D1 receptor; P2AR, Adrenergic beta 2 receptor; β1AR, Adrenergic beta 1 receptor; STE2, yeast αfactor receptor).

FIG. 4 shows the expression of GIR in adult rat tissues by Northern blot analysis (A) and RT-PCR Southern blot analysis (B):
A. Poly(A)+RNA (2 ug) from various rat tissues was hybridized with probes specific to rat GIR (A Upper Panel) and beta-actin (A Lower Panel) on a nylon membrane. The origin of each RNA is shown at the top, and the molecular mass of standard markers (in kilobases (kb)) is shown on the left. GIR expression is detected in brain. No detectable hybridization signal was seen in heart, spleen, lung, liver, muscle, kidney, and testis. The blot was stripped and rehybridized with a beta-actin probe.
B. B. A specific RT-PCR followed by Southern blot analysis was performed using RNA from 9 rat brain regions as following: Lane 1 to 9: transformed rat amygdala cells, caudate putanien, cerebellum, hippocampus, hypothalamus, nucleus accumbens, olfactory tube, prefrontal cortex, substantia nigra/VTA. The PCR primers are specific to the coding region of the rat GIR. The RT-PCR products were probed with a nested oligonucleotide primer specific for the rat GIR.

FIG. 5 shows the expression of GIR mRNA in the forebrain of the rat (A-D) and mouse (E). A. In rostral forebrain, GIR mRNA is expressed in medial prefrontal (mPFC) and piriform (PIR) cortex. B. Weak, but positive hybridization signal was observed in the shell of the nucleus accumbens (nACC). No signal was observed in the caudate-putamen (Cpu). C. The highest levels of GIR mRNA were detected in the nucleus of the lateral olfactory tract (LOT). Signal was also observed in several diencephalic nuclei, including the anteromedial thalamic nucleus (AM). Positive signal was seen in the hippocampus (HPC) at all levels. D. GIR mRNA expression was observed in CA1-CA4 subfields of the hippocampus (HPC) and in the dentate gyrus (DG). Dense hybridization signal was seen in zona incerta (ZI). GIR mRNA was also observed in selected hypothalamic nuclei, including the medial aspect of the arcuate nucleus (ARC). E. Localization of GIR mRNA in mouse forebrain. The same probe was used for panels A-E. Note extensive GIR mRNA in the caudate-putamen (CPu) and nucleus accumbens (NACC) of the mouse, as compared with the absence of expression in rat.

FIG. 6 shows the effects of amphetamine on rat GIR expression in prefrontal cortex quantitatively determined by competitive PCR analysis and on locomotion activities measured by cumulative numbers of crossovers. A. Representative competitive PCR image. B. GIR mRNA levels calculated from linear regression plot of the ratio plotted logarithmically against initial amount of input competitor DNA. Values are expressed as mean±SEM (n=6). Standard errors are represented by bars. [Bar with white and black diagonal stripes] denotes SAL-AMPM rats. ▪ denotes AMPH-AMPH rats. C. Cumulative numbers of crossovers measured during the 12-60 min and the 60-150 minute periods following challenge injection. Values are expressed as mean±SEM (n=6). Standard errors are represented by bars. [Bar with white and black diagonal stripes] denotes SAL-AMPH rats. ▪ denotes AMPH-AMPH rats. D. Bar graph represents the increased levels of GIR mRNA in prefrontal cortex after withdrawal from chronic amphetamine exposure in a separate experiment. Values are expressed as mean±SEM (n=6). Standard errors are represented by bars. □ denotes SAL rats. [Black bar with white dots] denotes AMPH rats.

FIG. 7 shows the nucleotide sequence of the human GIR.

FIG. 8 shows the nucleotide sequence of the human GIR isoform.

### DETAILED DESCRIPTION OF THE INVENTION

The present invention provides nucleotides and peptides for use in detecting central nervous system disorders.

Prior to setting forth the invention, it may be helpful to set forth definitions of certain terms to be used within the disclosure.

The term “agonists” is used herein to indicate any peptide or non-peptidyl compound that increases the biological activity of any of the polypeptides, receptors, nucleotides or other signal pathways of the subject invention.

“Associated impulsive-addictive-compulsive disorders” or “IACD” include specific disorders within the DSM-IV, The American Psychiatric Association, Washington, D.C., 1994, including Anxiety disorders, include Panic Disorder Without Agoraphobia, 300.01, Panic Disorder With Agoraphobia, 300.21, Agoraphobia Without History of Panic Disorder, 300.22, Specific Phobia, 300.29, Social Phobia, 300.23, Obsessive-Compulsive Disorder, 300.3, Posttraumatic Stress Disorder, 309.81, Acute Stress Disorder, 308.3, Generalized Anxiety Disorder, 300.02, Overanxious Disorder of Childhood, 300.02, Anxiety Disorder, 293.89, Substance Induced Anxiety Disorder, 293.89, Anxiety Disorder NOS, 300.00; Attention Deficit and Disruptive Behavior Disorders, including Attention-Deficit/Hyperactivity Disorder, Predominately Inattentive Type, 314.00, Attention-Deficit/Hyperactivity Disorder, Predominately Hyperactivity-Impulsive Type, 314.01 Attention-Deficit/Hyperactivity Disorder, Combined Type, 314.01, Attention-Deficit/Hyperactivity Disorder NOS, 314.9, Conduct Disorder, 312.8 Oppositional Defiant Disorder, 313.81, Disruptive Behavior Disorder NOS, 312.9; Bipolar Disorders including Bipolar I Disorder, 296.0x, 296.40, 296.4x, 296.6x, 296.5x, and 296.7, Bipolar II Disorder, 296.89, Cyclothymic Disorder, 301.13, Bipolar Disorder NOS, 296.80; Depressive Disorders including Major Depressive Disorder, Recurrent, 296.3, Dysthymic Disorder, 300.4, Depressive Disorder NOS, 311, Major Depressive Disorder, Single Episode, 296.2; Eating Disorders including Bulimia Nervosa, Nonpurging Type, 307.51, Bulimia Nervosa, Purging Type, 307.51, Anorexia Nervosa, 307.1, Eating Disorder NOS 307.50; Impulse Control Disorders including Intermittent Explosive Disorder, 312.34, Kleptomania, 312.32, Pyromania, 312.23, Pathological Gambling, 312.31, Trichotillomania, 312.39, Impulse Control Disorder NOS, 312.30; Personality Disorders including Antisocial Personality Disorder, 301.7, Avoidant Personality Disorder, 301.82, Obsessive-Compulsive Personality Disorder, 301.4, Schizoid Personality Disorder, 301.20; Schizophrenia including Paranoid Type, 295.30, Disorganized Type, 295.10, Catatonic Type, 295.20, Undifferentiated Type, 295.90, Residual Type, 295.60, Schizoaffective Disorder, 295.70, Schizophreniform Disorder, 295.40; Sleep Disorders including Primary Sleep Disorders such as Dyssomnias which include Primary Insomnia 307.42, Primary Hypersomnia 307.44, Narcolepsy 347, Circadian Rhythm Sleep Disorder, 307.45, Dyssomnia NOS 307.47, Parasomnias which include Nightmare Disorder 307.47, Sleep Terror Disorder 307.46, Sleepwalking Disorder 307.46, Parasomnia NOS 307.47, Sleep Disorders Related to Another Mental Disorder which include Insomnia Related to Axis I or Axis II disorder 307.42, Hypersomnia Related to Axis I or Axis II disorder 307.44, Other Sleep Disorders, Substance Induced Sleep Disorder; Substance Use Disorders including Alcohol Related Disorders such as Alcohol-Induced Psychotic Disorder, with delusions, 291.5, Alcohol Abuse, 305.00, Alcohol Intoxication, 303.00, Alcohol Withdrawal, 291.8, Alcohol Intoxication Delirium, 291.0, Alcohol Withdrawal Delirium, 291.0, Alcohol-Induced Persisting Dementia, 291.2, Alcohol-Induced Persisting Amnestic Disorder, 291.1, Alcohol Dependence, 303.90, Alcohol-Induced Psychotic Disorder, with hallucinations, 291.3, Alcohol-Induced Mood Disorder, 291.8, Alcohol-Induced Anxiety Disorder, 291.8, Alcohol-Induced Sexual Dysfunction, 291.8, Alcohol-Induced Sleep Disorder, 291.8, Alcohol-Related Disorder NOS, 291.9, Alcohol Intoxication, 303.00, Alcohol Withdrawal, 291.S, Nicotine Related Disorders which include Nicotine Dependence, 305.10, Nicotine Withdrawal, 292.0, Nicotine-Related Disorder NOS, 292.9, Amphetamine Related Disorders which include Amphetamine Dependence, 304.40, Amphetamine Abuse, 305.70, Amphetamine Intoxication, 292.89, Amphetamine Withdrawal, 292.0, Amphetamine Intoxication Delirium, 292.81, Amphetamine-Induced Psychotic Disorder with delusions, 292.11, Amphetamine-Induced Psychotic Disorders with hallucinations, 292.12, Amphetamine-Induced Mood Disorder, 292.84, Amphetamine-Induced Anxiety Disorder, 292.89, Amphetamine-Induced Sexual Dysfunction, 292.89, Amphetamine-Induced Sleep Disorder, 292.89, Amphetamine Related Disorder NOS, 292.9, Amphetamine Intoxication, 292.89, Amphetamine Withdrawal, 292.0, Cannabis Related Disorders which include Cannabis Dependence, 304.30, Cannabis Abuse, 305.20, Cannabis Intoxication, 292.89, Cannabis Intoxication Delirium, 292.81, Cannabis-Induced Psychotic Disorder, with delusions, 292.11, Cannabis-Induced Psychotic Disorder with hallucinations, 292.12, Cannabis-Induced Anxiety Disorder, 292.89, Cannabis Related Disorder NOS, 292.9, Cannabis Intoxication, 292.89, Cocaine Related Disorders which include Cocaine Dependence, 304.20, Cocaine Abuse, 305.60, Cocaine Intoxication, 292.89, Cocaine Withdrawal, 292.0, Cocaine Intoxication Delirium, 292.81, Cocaine-Induced Psychotic Disorder with delusions, 292.11, Cocaine-Induced Psychotic Disorders with hallucinations, 292.12, Cocaine-Induced Mood Disorder, 292.84, Cocaine-Induced Anxiety Disorder, 292.89, Cocaine-Induced Sexual Dysfunction, 292.89, Cocaine-Induced Sleep Disorder, 292.89, Cocaine Related Disorder NOS, 292.9, Cocaine Intoxication, 292.89, Cocaine Withdrawal, 292.0; Hallucinogen Use Disorders which include Hallucinogen Dependence, 304.50, Hallucinogen Abuse, 305.30, Hallucinogen Intoxication, 292.89, Hallucinogen Withdrawal, 292.0, Hallucinogen Intoxication Delirium, 292.81, Hallucinogen-Induced Psychotic Disorder with delusions, 292.11, Hallucinogen-Induced Psychotic Disorders with hallucinations, 292.12, Hallucinogen-Induced Mood Disorder, 292.84, Hallucinogen-Induced Anxiety Disorder, 292.89, Hallucinogen-Induced Sexual Dysfunction, 292.89, Hallucinogen-Induced Sleep Disorder, 292.89, Hallucinogen Related Disorder NOS, 292.9, Hallucinogen Intoxication, 292.89, Hallucinogen Persisting Perception Disorder (Flashbacks), 292.89; Inhalant Related Disorders which include Inhalant Dependence, 304.60, Inhalant Abuse, 305.90, Inhalant Intoxication, 292.89, Inhalant Intoxication Delirium, 292.81, Inhalant-Induced Psychotic Disorder, with delusions, 292.11, Inhalant-Induced Psychotic Disorder with hallucinations, 292.12, Inhalant-Induced Anxiety Disorder, 292.89, Inhalant Related Disorder NOS, 292.9, Inhalant Intoxication, 292.89; Opioid Related Disorders which include Opioid Dependence, 304.00, Opioid Abuse, 305.50, Opioid Intoxication, 292.89, Opioid Intoxication Delirium, 292.81, Opioid-Induced Psychotic Disorder, with delusions, 292.11, Opioid-Induced Psychotic Disorder with hallucinations, 292.12, Opioid-Induced Anxiety Disorder, 292.89, Opioid Related Disorder NOS, 292.9, Opioid Intoxication, 292.S9, Opioid Withdrawal, 292.0; Polysubstance Related Disorders which include Polysubstance Dependence, 304.80; Tic Disorders which include Tourette's Disorder, 307.23, Chronic Motor or Vocal Tic Disorder 307.22, Transient Tic Disorder 307.21, Tic Disorder NOS 307.20, Stuttering 307.0, Autistic Disorder, 299.00, and Somatization Disorder 300.81. Additionally, other RDS disorders are defined as would be known to one of skill in the art.

The term “antagonist” is used throughout this application to indicate any peptide or nonpeptidyl compound which decreases the biological activity of any of the polypeptides, receptors, nucleotides or other signal pathways of the subject invention.

“Antibody” refers to a molecule that is a member of a family of glycosylated proteins called immunoglobulins that can specifically combine with an antigen. Such an antibody combines with its antigen by a specific immunologic binding interaction between the antigenic determinant of the antigen and the antibody-combining site of the antibody.

“Antigen” has been used historically to designate an entity that is bound by an antibody, and also to designate the entity that induces the production of the antibody. More current usage limits the meaning of antigen to that entity bound by an antibody, whereas the word “immunogen” is used for the entity that induces antibody production. Where an entity discussed herein is both immunogenic and antigenic, it will generally be termed an antigen.

“Biological activity” refers to a function or set of activities performed by a molecule in a biological context (i.e., in an organism or an in vitro facsimile thereof).

A DNA “coding sequence” or a “nucleotide sequence encoding” a particular protein, is a DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, procaryotic sequences, cDNA from eucaryotic mRNA, genomic DNA sequences from eucaryotic (e.g., mammalian) DNA, and even synthetic DNA sequences. A transcription termination sequence will usually be located 3′ to the coding sequence.

“Digestion” of DNA refers to catalytic cleavage of the DNA with a restriction enzyme that acts only at certain sequences in the DNA. The various restriction enzymes used herein are commercially available and their reaction conditions, cofactors and other requirements were used as would be known to the ordinarily skilled artisan.

A “DNA construct” is a DNA molecule, or a clone of such a molecule, either single- or double-stranded that has been modified through human intervention to contain segments of DNA combined and juxtaposed in a manner that as a whole would not otherwise exist in nature.

A “double-stranded DNA molecule” refers to the polymeric form of deoxyribonucleotides (bases adenine, guanine, thymine, or cytosine) in a double-stranded helix, both relaxed and supercoiled. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having the sequence homologous to the mRNA).

“ELISA” refers to an enzyme-linked immunosorbent assay that employs an antigen or antibody bound to a solid phase and an enzyme-antibody or enzyme-antigen conjugate to detect and quantify the amount of antigen or antibody present in a sample. A description of the ELISA technique is found in U.S. Pat. Nos. 3,654,090, 3,850,752, and 4,016,043, which are incorporated herein by reference.

The term “encoding” refers generally to the sequence information being present in a translatable form, usually operably linked to a promoter. A sequence is operably linked to a promoter when the functional promoter enhances transcription or expression of that sequence. An anti-sense strand is considered to also encode the sequence, since the same informational content is present in a readily accessible form, especially when linked to a sequence that promotes expression of the sense strand. The information is convertible using the standard, or a modified, genetic code.

An “isolated” nucleic acid is a nucleic acid, e.g., an RNA, DNA, or a mixed polymer, which is substantially separated from other DNA sequences which naturally accompany a native human sequence, e.g., ribosomes, polymerases, and many other human genome sequences. An “isolated” or “substantially pure” nucleic acid (e.g., an RNA, DNA or a mixed polymer) is one which is substantially separated from other cellular components which naturally accompany a native human sequence or protein, e.g., ribosomes, polymerases, many other human genome sequences and proteins. The term embraces a nucleic acid sequence or protein which has been removed from its naturally occurring environment, and includes recombinant or cloned DNA isolates and chemically synthesized analogs or analogs biologically synthesized by heterologous systems.

“GIR Allele” refers to normal alleles of the GIR locus as well as alleles carrying variations that predispose individuals to develop diseases of the central nervous system. Such predisposing alleles are also called “GIR susceptibility alleles”.

“GIR Locus,” “GIR Gene,” “GIR Nucleic Acids” or “GIR Polynucleotide” each refer to polynucleotides, all of which are in the glucocorticoid induced receptor (GIR) region, that are likely to be expressed in normal tissue, certain alleles of which predispose an individual to develop diseases of the central nervous system. The GIR locus is intended to coding sequences, intervening sequences and regulatory elements controlling transcription and/or translation. The GIR locus is intended to include all allelic variations of the DNA sequence. These terms, when applied to a nucleic acid, refer to a nucleic acid which encodes a glucocorticoid induced receptor polypeptide, fragment, homolog or variant, including, e.g., protein fusions or deletions.

The glucocorticoid induced receptor (GIR) bears closest amino acid identity with the neuropeptide Y (NPY) family of G-protein coupled receptors (˜38%).

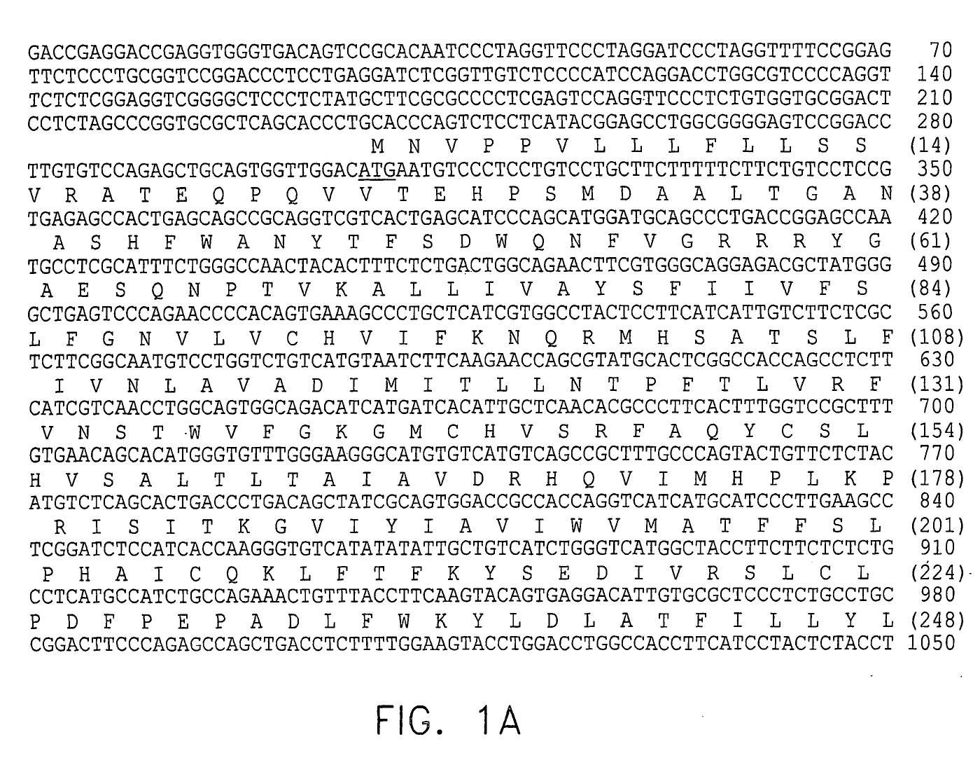

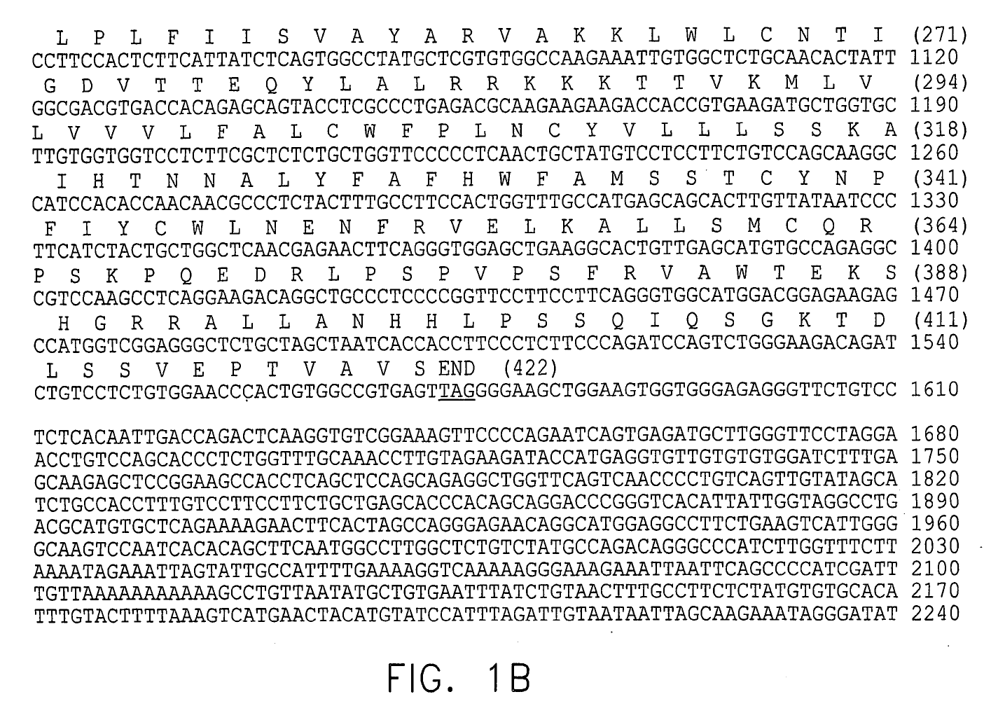

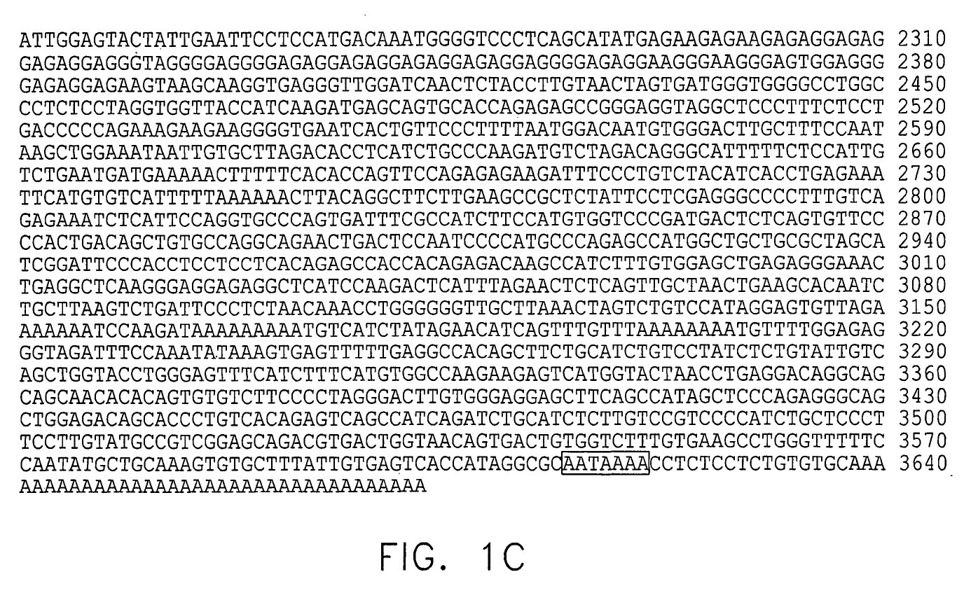

The nucleic acids of the present invention will possess a sequence that is either derived from, or substantially similar to a natural rat GIR-encoding gene or one having substantial homology with a natural GIR-encoding gene or a portion thereof. The coding sequence for a rat GIR polypeptide (SEQ ID NO:1) is shown in FIG. 1, along with the amino acid sequence (SEQ ID NO:2).

The present invention also provides for the nucleotide and amino acid sequences that possess a sequence that is either derived from, or substantially similar to a natural human GIR gene and an isoform of the human GIR gene or one having substantial homology with a natural human GIR gene and an isoform of the human GIR gene or portions thereof. The coding sequence for a human GIR polypeptide (SEQ ID NO:3) is shown in FIG. 7, along with the amino acid sequence (SEQ ID NO:4). The coding sequence for a human isoform GIR polypeptide (SEQ ID NO:5) is shown in FIG. 8, along with the amino acid sequence (SEQ ID NO:6).

As used herein, the terms “GIR locus,” “GIR allele” and “GIR region” all refer to the double-stranded DNA comprising the locus, allele, or region, as well as either of the single-stranded DNAs comprising the locus, allele or region. As used herein, a “portion” of the GIR locus or region or allele is defined as having a minimal size of at least about eight nucleotides, or preferably about 15 nucleotides, or more preferably at least about 25 nucleotides, and may have a minimal size of at least about 40 nucleotides.

“GIR protein” or “GIR polypeptide” refer to a protein or polypeptide encoded by the GIR locus, variants or fragments thereof. The term “polypeptide” refers to a polymer of amino acids and its equivalent and does not refer to a specific length of the product; thus, peptides, oligopeptides and proteins are included within the definition of a polypeptide. Included within the definition are, for example, polypeptides containing one or more analogs of an amino acid (including, for example, unnatural amino acids, etc.), polypeptides with substituted linkages as well as other modifications known in the art, both naturally and non-naturally occurring. Ordinarily, such polypeptides will be at least about 50% homologous to the native GIR sequence, preferably in excess of about 90%, and more preferably at least about 95% homologous. Also included are proteins encoded by DNA which hybridize under high or low stringency conditions, to GIR-encoding nucleic acids and closely related polypeptides or proteins retrieved by antisera to the GIR protein(s). The GIR polypeptide of the present invention also includes conservative variations of the polypeptide sequence. The term “conservative variation” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine glutamic for aspartic acids, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

“Oligonucleotides” refers to either a single stranded polydeoxynucleotide or two complementary polydeoxynucleotide strands that may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

“Probes” are polynucleotide sequences that form a stable hybrid with that of the target sequence, under stringent to moderately stringent hybridization and wash conditions. Polynucleotide polymorphisms associated with GIR alleles that predispose individuals to develop certain diseases of the central nervous system are detected by hybridization with a probe. If it is expected that the probes will be perfectly complementary to the target sequence, stringent conditions will be used. Hybridization stringency may be lessened if some mismatching is expected, for example, if variants are expected with the result that the probe will not be completely complementary. Conditions are chosen which rule out nonspecificladventitious bindings, that is, which minimize noise. Since such indications identify neutral DNA polymorphisms as well as mutations, these indications need further analysis to demonstrate detection of a GIR susceptibility allele. Probes for GIR alleles may be derived from the sequences of the GIR region or its cDNAs. The probes may be of any suitable length, which span all or a portion of the GIR region, and which allow specific hybridization to the GIR region. If the target sequence contains a sequence identical to that of the probe, the probes may be short, e.g., in the range of about 8 to about 30 base pairs, since the hybrid will be relatively stable under even stringent conditions. If some degree of mismatch is expected with the probe, i.e., if it is suspected that the probe will hybridize to a variant region, a longer probe may be employed which hybridizes to the target sequence with the requisite specificity. The probes will include an isolated polynucleotide attached to a label or reporter molecule and may be used to isolate other polynucleotide sequences, having sequence similarity by standard methods. Other similar polynucleotides may be selected by using homologous polynucleotides.

“Protein modifications or fragments” are provided by the present invention for GIR polypeptides or fragments thereof which are substantially homologous to primary structural sequence but which include, e.g., in vivo or in vitro chemical and biochemical modifications or which incorporate unusual amino acids. Such modifications include, for example, acetylation, carboxylation, phosphorylation, glycosylation, ubiquitination, labeling, e.g., with radionuclides, and various enzymatic modifications, as will be readily appreciated by those well skilled in tile art.

“Recombinant polypeptides” refer to polypeptides produced by recombinant DNA techniques; i.e., produced from cells transformed by an exogenous DNA construct encoding the desired polypeptide. “Synthetic” polypeptides are those prepared by chemical synthesis.

A “Restriction Fragment” is a DNA molecule produced by digestion with a restriction endonuclease. Any given genome will be digested by a particular restriction endonuclease into a discrete set of restriction fragments.

“Restriction Fragment Length Polymorphism (RFLP) refers to when the genomic DNA of two individuals in a population will differ in sequence at many sites either as a result of change in bases or insertions or deletions of sequences. When these differences occur in the recognition site for a restriction endonuclease, then a polymorphism in the length of restriction fragments produced by digestion of the DNA of the two individuals will result.

“Substantial homology or similarity”. A nucleic acid or fragment thereof is “substantially homologous” (“or substantially similar”) to another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases. Alternatively, substantial homology or (similarity) exists when a nucleic acid or fiagment thereof will hybridize to another nucleic acid (or a complementary strand thereof) under selective hybridization conditions, to a strand, or to its complement. Selectivity of hybridization exists when hybridization which is substantially more selective than total lack of specificity occurs. Typically, selective hybridization will occur when there is at least about 55% homology over a stretch of at least about 14 nucleotides, preferably at least about 65%, more preferably at least about 75%, and most preferably at least about 90%. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Homology, for polypeptides, is typically measured using sequence analysis software. Protein analysis software matches similar sequences using measure of homology assigned to various substitutions, deletions and other modifications. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine.

A polypeptide “fragment,” “portion” or “segment” is a stretch of amino acid residues of at least about five to seven contiguous amino acids, often at least about seven to nine contiguous amino acids, typically at least about nine to 13 contiguous amino acids and, most preferably, at least about 20 to 30 or more contiguous amino acids. The polypeptides of the present invention, if soluble, may be coupled to a solid-phase support, e.g., nitrocellulose, nylon, column packing materials (e.g., Sepharose beads), magnetic beads, glass wool, plastic, metal, polymer gels, cells, or other substrates. Such supports may take the form, for example, of beads, wells, dipsticks, or membranes.

“Target region” refers to a region of the nucleic acid that is amplified and/or detected. The term “target sequence” refers to a sequence with which a probe or primer will form a stable hybrid under desired conditions.

It is widely held that various psychological disorders are linked by a common biological determinant in the brain that provides pleasure or reward in the process of certain behavior. Certain inborn chemical imbalances that alter the intracellular signaling in the limbic reward regions could alter an individual's feeling of well being causing anxiety, anger or craving for a substance that can alleviate negative reactions. This chemical imbalance manifests itself in one or more behavioral disorders. As used herein, the term “stress reward disorder” refers to a variety of associated impulsive-addictive-compulsive disorders (“IACD”) including polysubstance abuse, nicotine addiction, attention deficit hyperactivity disorder, memory disorders, aggression, post-traumatic stress syndrome, PMS, learning disorders, manic symptoms, phobias, panic attacks, sleep disorders, anxiety disorders, mood disorders, eating disorders, psychotic disorders, impulse control disorders, personalities disorders, acute stress disorder, generalized anxiety disorder, obesity, Tourette Syndrome, pathological gambling as well as other behavior anomalies.

The present invention provides a method of predicting a patient's likelihood of developing a stress reward disorder comprising (a) obtaining DNA from a human subject; and (b) detecting in said DNA a human glucocorticoid-induced receptor gene wherein the gene indicates a potential susceptibility to an addiction reward disorder. Detecting the human GIR receptor gene is generally performed by subjecting the DNA of the subject to digestion by restriction enzyme, hybridizing the DNA to a labeled probe specifically binding to a GIR gene allele of the human GIR receptor, and determining the presence of the allele of the human GIR receptor.

The probe is generally a recombinant cDNA or a fragment thereof. Preferably, the DNA is separated by electrophoresis according to size. The stress reward disorder is a disorder generally selected from the group consisting of polysubstance abuse, drug addiction, nicotine addiction, alcohol addiction and cocaine addiction. The detection step generally involves RFLP or PASA.

The present invention also provides a method of detecting a genetic potential susceptibility to stress reward disorder in a human subject comprising obtaining DNA from the subject; subjecting the DNA of the subject to digestion by a restriction enzyme; hybridizing the DNA to a labeled probe specifically binding to the nucleotide sequences of SEQ ID NO:1, SEQ ID NO:3, or SEQ ID NO:5; and determining the presence of the GIR receptor. The present invention provides methods of detecting the presence of a stress reward disorder susceptibility locus in an individual by analyzing a sample of DNA from the individual for the presence of a DNA polymorphism of the GIR gene sequence wherein the polymorphism is associated with stress reward disorder. The methods can be by genetically diagnosing an addiction reward disorder in an individual comprising analyzing a DNA sample from an individual for the presence of a DNA polymorphism associated with addiction reward disorder wherein the DNA polymorphism is in the GIR gene region and the presence of the DNA polymorphism is an indication that the individual has addiction reward disorder.

The present invention provides a method of treating an individual phenotypically diagnosed with an addiction reward disorder comprising analyzing a DNA sample from an individual phenotypically diagnosed with an addiction reward disorder for the presence or absence of a DNA polymorphism associated with addiction reward disorder wherein the DNA polymorphism is located within a region of the GIR gene allele and wherein the presence of a DNA polymorphism associated with addiction reward disorder confirms the diagnosis; and selecting a treatment plan that is most effective for individuals phenotypically diagnosed as having addiction reward disorder.

The present invention also provides a method of diagnosis of GIR receptor sensitization in a mammalian subject by obtaining a DNA sample from a subject; amplifying the DNA sample (this can alternatively be by reverse transcribing an RNA sample from the patient into DNA and then amplifying the DNA); and analyzing the amplified DNA of step (a) to determine whether the sample comprises at least one disease causing sequence abnormality with respect to the GIR nucleotide sequences as set forth in SEQ ID NO: 1, SEQ ID NO:3, or SEQ ID NO:5, or a sequence encoding the GIR amino acid sequence as set forth in SEQ ID NO: 2, SEQ ID NO:4, or SEQ ID NO:6, such abnormality leading to a decrease (or increase) in the GIR activity and being indicative of a GIR disorder.

The present invention also provides a method for identifying a chemical compound which specifically binds to a mammalian or rodent GIR receptor which comprises contacting cells transfected with DNA encoding and expressing the mammalian or rodent GIR receptor or a membrane fraction from such cells with the compound under conditions suitable for binding and detecting specific binding of the chemical compound to the receptor wherein such cells or membrane fraction do not normally express the receptor and wherein the receptor has an amino acid sequence substantially similar to the amino acid sequence shown in SEQ ID NO: 2, SEQ ID NO:4, or SEQ ID NO:6.

The present invention also provides a method for determining whether a chemical compound is a mammalian GIR receptor agonist which comprises contacting cells transfected with and expressing DNA encoding a mammalian GIR receptor or a membrane fraction from such cells with the compound under conditions permitting the activation of the GIR receptor and detecting an increase in receptor activity so as to thereby determine whether the compound is a receptor agonist wherein the receptor has an amino acid sequence of substantially similar to the amino acid sequence shown in SEQ ID NO: 2, SEQ ID NO:4, or SEQ ID NO:6.

The present invention also provides a method for determining whether a chemical compound is a mammalian GIR receptor antagonist which comprises contacting cells transfected with and expressing DNA encoding a mammalian GIR receptor or a membrane fraction from such cells with the compound under conditions permitting the activation of the GIR receptor and detecting an increase in receptor activity so as to thereby determine whether the compound is a receptor antagonist wherein the receptor has an amino acid sequence of substantially similar to the amino acid sequence shown in SEQ ID NO: 2, SEQ ID NO:4, or SEQ ID NO:6. Generally, the cells are mammalian cells. Preferably, the mammalian cells are non-neuronal in origin.

The present invention also provides a method for determining whether a chemical compound specifically binds to and activates a mammalian GIR receptor by contacting cells transfected with DNA encoding and expressing on their cell surface the GIR receptor and producing a second messenger response upon activation of the receptor with the chemical compound under conditions suitable for activation of the receptor and measuring the second messenger response in the presence and in the absence of the chemical compound. A change in the second messenger response in the presence of the chemical compound indicates that the compound activates the receptor and wherein such cells do not normally express the GIR receptor. The receptor has an amino acid sequence substantially similar to the amino acid sequence shown in SEQ ID NO: 2, SEQ ID NO:4, or SEQ ID NO:6.

The present invention also provides a method for determining whether a chemical compound specifically binds to and inactivates a mammalian GIR receptor which comprise contacting cells transfected with DNA encoding and expressing on their cell surface the GIR receptor and producing a second messenger response upon activation of the receptor with the chemical compound under conditions suitable for inactivation of the receptor and measuring the second messenger response in the presence and in the absence of the chemical compound. A change in the second messenger response in the presence of the chemical compound indicates that the compound inactivates the receptor and wherein such cells do not normally express the GIR receptor and the receptor has an amino acid sequence substantially similar to the amino acid sequence shown in SEQ ID NO: 2, SEQ ID NO:4, or SEQ ID NO:6. Preferably, the second messenger response comprises intra-cellular calcium levels.

The present invention also provides a method for screening a plurality of chemical compounds not known to bind a mammalian GIR receptor to identify a compound that specifically binds to the receptor by contacting cells transfected with and expressing DNA encoding the GIR receptor or a membrane fraction from such cells with a compound known to bind specifically to the GIR receptor; contacting or a membrane fraction identical to those contacted in step (a) with the plurality of compounds not known to bind specifically to the receptor under conditions permitting binding of compounds known to bind the receptor; determining whether the binding of the compound known to bind the receptor is reduced in the presence of one or more of the compounds within the plurality of compounds relative to the binding of the compound in the absence of the plurality of compounds; and separately determining the binding to the receptor of each compound included in the plurality of compounds so as to thereby identify the compound or compounds which specifically binds to the GIR receptor and wherein the mammalian GIR receptor has an amino acid sequence substantially similar to the amino acid sequence shown in SEQ ID NO: 2, SEQ ID NO:4, or SEQ ID NO:6.

The present invention also provides a method for screening a plurality of chemical compounds to identify a compound that activates the GIR receptor by contacting cells transfected with and expressing the GIR receptor with the plurality of compounds to activate the GIR receptor or a membrane fraction from such cells under conditions permitting activation of the receptor and determining whether the activity of the receptor is increased in the presence of one or more of the compounds within the plurality of compounds and separately determining which compounds activate the GIR receptor so as to thereby identify the compound or compounds that activate the receptor.

The present invention also provides a kit for use in genetically detecting potential susceptibility to stress reward disorders in a human subject. Such a kit generally contains a container including a restriction enzyme capable of cleaving a human GIR receptor gene; and a container including a hybridization probe for detecting a human GIR receptor gene allele whose presence indicates susceptibility to addiction reward disorder. Preferably, the restriction enzyme is Taq I. Alternatively, the kit may contain a container with PASA primers specifically binding GIR receptor alleles characterizing susceptibility to stress reward disorder; and a second container comprising ingredients for PCR amplification of specific GIR gene alleles.

The activity of a G-protein coupled receptor such as the polypeptides disclosed herein may be measured using any of a variety of functional assays in which activation of the receptor in question results in an observable change in the level of some second messenger system including but not limited to adenylate cyclase, calcium mobilization, arachidonic acid release, ion channel activity, inositol phospholipid hydrolysis or guanylyl cyclase. Heterologous expression systems utilizing appropriate host cells to express the nucleic acid of the subject invention are used herein to obtain the desired second messenger coupling. Receptor activity may also be assayed in an oocyte expression system.

It is possible that the mammalian GIR receptor contains introns and additionally the possibility exists that additional introns could exist in coding or non-coding regions. In addition, spliced forms of mRNA may encode additional amino acids either upstream of the currently defined starting codon or within the coding region. Furthermore, the existence and use of alternative exons is possible wherein the mRNA may encode different amino acids within the region comprising the exon. In addition, single amino acid substitutions may arise within the mechanism of RNA editing such that the amino acid sequence of the express protein is different than that encoded by the original gene. Such variants may exhibit pharmacological properties differing from the polypeptide encoded by the original gene.

This invention provides for the above-referenced isolated nucleic acid wherein the nucleic acid is DNA. In one embodiment, the DNA is cDNA. In another embodiment, the DNA is genomic DNA. In a further embodiment, the nucleic acid molecule is RNA. Method for production and manipulation of nucleic acid molecules are well known in the art. This invention further provides nucleic acid which is degenerate with respect to the DNA comprising the coding sequence. This invention also encompasses DNAs and cDNAs which encode amino acid sequences that differ from those of the polypeptides of this invention but which should not produce phenotypic changes. Alternatively this invention also encompasses DNAs, cDNAs and RNAs which hybridize to the DNA, cDNA and RNA of the subject invention. Hybridization methods are well known to those of skill in the art.

The nucleic acids of the present invention also include nucleic acid molecules coding for polypeptide analogs, fragments or derivatives of antigenic polypeptides which differ from naturally occurring forms in terms of the identity or location of one or more amino acid residues such as where deletion analogs contain less than all of the residues specified for the protein where substitution analogs have one or more residues specified replaced by other residues and addition analogs where one or more amino acid residues is added to a terminal remedial portion of the polypeptide whereby such derivative shares some or all properties of the naturally occurring forms. These molecules include the incorporation of codons that are preferred for expression by selected hosts. The provision of sites for cleavage by restriction endonuclease enzymes and the provision for additional initial terminal or intermediate DNA sequences that facilitate construction of readily expressed vectors.

The modified polypeptides of this invention may be transfected into cells either transiently or stably using methods well known in the art. The present invention also provides for binding assays using the modified polypeptides in which the polypeptide is expressed either transiently or in stable cell lines. The present invention also encompasses compounds identified using a modified polypeptide in a binding assay such as the assays described herein.

In one embodiment of the present invention, the mammalian GIR receptor is a human GIR receptor. In another embodiment, the mammalian GIR receptor is a rodent GIR receptor. In another embodiment, the human GIR receptor has an amino acid sequence substantially the same as the amino acid sequence shown in SEQ ID NO: 2, SEQ ID NO:4, or SEQ ID NO:6. In another embodiment, the human GIR receptor has an amino acid sequence identical to the amino acid sequence shown in SEQ ID NO: 2, SEQ ID NO:4, or SEQ ID NO:6. This invention also provides an isolated nucleic acid encoding a modified mammalian GIR receptor that differs from a mammalian GIR receptor by having one or more amino acid deletion replacement or addition. This invention further provides for a purified mammalian GIR receptor protein.

This invention provides for a vector comprising the nucleic acid of a mammalian GIR receptor. In a further embodiment, the mammalian GIR receptor is a human GIR receptor. In one embodiment the vector is adapted for expression in a bacterial cell that comprises the regulatory elements necessary for expression of the nucleic acid in the bacterial cell operatively linked to the nucleic acid encoding a mammalian GIR receptor to permit expression thereof. In another embodiment, the vector is adapted for expression in an amphibian cell that comprises the regulatory elements necessary for expression of the nucleic acid in the amphibian cell operatively linked to the nucleic acid encoding a mammalian GIR receptor to permit expression. In another embodiment, the vector is adapted for expression in a yeast cell that comprises the regulatory elements necessary for expression of the nucleic acid in the yeast cell operatively linked to the nucleic acid encoding a mammalian GIR receptor to permit expression. In a further embodiment, the vector is a plasmid. Preferably the plasmid comprises the regulatory elements necessary for expression of DNA in a mammalian cell operatively linked to DNA encoding for the mammalian GIR receptor so as to allow expression.

The present invention further provides for any vector or plasmid that comprises modified untranslated sequences which are beneficial for expression in desired host cells or for use in binding or functional assays. For example, a vector or plasmid with untranslated sequences of varying lengths may express amounts of the polypeptide depending upon the host cell used. In another embodiment, the vector or plasmid comprises the coding sequences of the polypeptide and the regulatory elements necessary for expression in the host cell. The present also provides for a cell comprising the vector comprising a nucleic acid encoding for the mammalian GIR receptor. In one embodiment, the cell is a non-mammalian cell. In another embodiment, the non-mammalian cell is a Xenopus oocyte cell. In another embodiment, the cell is a mammalian cell. Preferably the mammalian cell is NIH-3T3 cells or CHO cells.

The present invention also provides for a nucleic acid probe comprising at least 15 nucleotides wherein the probe specifically hybridizes with a nucleic acid encoding for a mammalian GIR receptor wherein the probe further has a unique sequence corresponding to a sequence present within one of the two strands of the nucleic acid which encode for the mammalian GIR receptor. Preferably the nucleic acid probe comprises at least 15 nucleotides wherein the probe specifically hybridizes with the nucleic acid encoding a mammalian GIR receptor and wherein the probe has a unique sequence corresponding to a sequence present within the nucleic acid sequence shown in FIG. 1. (Sequence ID Number 1 or the reverse complement thereof.) In one embodiment, the nucleic acid is DNA. In a further embodiment, the nucleic acid is RNA.

The present invention also provides for a nucleic acid probe comprising a nucleic acid molecule of at least 15 nucleotides which is complementary to a unique fragment of the sequence of a nucleic acid molecule encoding a mammalian GIR receptor. Also provided is a nucleic acid probe comprising a nucleic acid molecule of at least 15 nucleotides complementary to the antisense sequence of a unique fragment of the sequence of a nucleic acid molecule encoding a mammalian GIR receptor.

As used herein, the term “specifically hybridizing” means the ability of a nucleic acid molecule to recognize a nucleic acid sequence complementary to its own and to form double helical segments through hydrogen bonding between complementary based pairs.

Nucleic acid probe technology is well known to those skilled in the art who will readily appreciate that such probes may vary greatly in length and may be labeled with a detectable label such as a radioisotope fluorescent label or electron resonance label to facilitate detection of the probe. DNA probe molecules may be produced by insertion of a DNA molecule that encodes the polypeptides of the present invention into suitable vectors such as plasmids or bacteriophages followed by transforming into suitable bacterial host cells, replication in the transformed bacterial host cells and harvesting of the DNA probes using methods well known in the art. Alternatively, probes may be generated chemically from DNA synthesizers.

RNA probes may be generated by inserting the DNA molecule that encodes the polypeptides of the present invention downstream of a bacteriophage promoter such as T3T7 or SP6. Large amounts of RNA probe may be produced by incubating the labeled nucleotides with the fragment where it contains an upstream promoter in the presence of the appropriate RNA preliminaries.

The present invention provides for antisense nucleotides having a sequence capable of specifically hybridizing to the RNA encoding for a mammalian GIR receptor so as to prevent translation of the RNA. Also provided is an antisense nucleotide having a sequence capable of specifically hybridizing to the genomic DNA encoding a mammalian GIR receptor. In one embodiment, the antisense oligonucleotide comprises chemically modified nucleotides or nucleotide analogs.

In another embodiment, the present invention provides for an antibody capable of binding to a mammalian GIR receptor. In one embodiment, the mammalian GIR receptor is a human GIR receptor. Also provided for is an agent capable of competitively inhibiting the binding of the antibody to a mammalian GIR receptor. In a further embodiment, the antibody is a monoclonal antibody or antisera.

The present invention also provides for a pharmaceutical composition comprising an amount of an oligonucleotide capable of passing through a cell membrane and which is effective at reducing expression of a mammalian GIR receptor and a pharmaceutically acceptable carrier. In one embodiment, the oligonucleotide is coupled to a substance that inactivates mRNA. In another embodiment, the compound which inactivates mRNA is a ribosome. In another embodiment, pharmaceutically acceptable carrier comprises a structure which binds to a mammalian GIR receptor on a cell capable of being taken up by the cells after binding to the structure. In a further embodiment, the pharmaceutically acceptable carrier is capable of binding to a mammalian GIR receptor that is specific for a selected cell type. In another embodiment, the present invention provides a pharmaceutical composition comprising an amount of an antibody effective at blocking binding of a ligand to a human GIR receptor along with a pharmaceutically acceptable carrier. As used herein, “pharmaceutically carriers” means any of the standard pharmaceutically acceptable carrier. Examples include, but are not limited to, phosphate buffered saline, physiological saline, emulsions such as oil and water emulsions and water.

The present invention also provides for a transgenic non-human mammal expressing the DNA encoding for a mammalian GIR receptor. This invention also provides for a transgenic nonhuman mammal comprising a homologous recombination knock out of the native mammalian GIR receptor.

This invention further provides for a transgenic, nonhuman mammal whose genome comprises antisense DNA complementary to the DNA encoding a mammalian GIR receptor so placed within the genome as to be transcribed into antisense mRNA which is complementary to mRNA encoding a mammalian GIR receptor and which hybridizes to mRNA encoding a mammalian GIR receptor, thereby reducing its translation. In an embodiment, the DNA encoding a mammalian GIR receptor additionally comprises an inducible promoter. In a further embodiment, the DNA encoding a mammalian GIR receptor additionally comprises tissue specific regulatory elements. In another embodiment, the transgenic, nonhuman mammal is a mouse.

Animal model systems which elucidate the physiological and behavioral roles of the polypeptides of this invention are produced by creating transgenic animals in which the activity of the polypeptide is either increased or decreased, or the amino acid sequence of the expressed polypeptide is altered, by a variety of techniques. Examples of these techniques include, but are not limited to: 1) Insertion of normal or mutant versions of DNA encoding the polypeptide, by microinjection, electroporation, retroviral transfection or other means well known to those skilled in the art, into appropriate fertilized embryos in order to produce a transgenic animal or 2) Homologous recombination of mutant or normal, human or animal versions of these genes with the native gene locus in transgenic animals to alter the regulation of expression or the structure of these polypeptide sequences. The technique of homologous recombination is well known in the art. It replaces the native gene with the inserted gene and so is useful for producing an animal that cannot express native polypeptides but does express, for example, an inserted mutant polypeptide, which has replaced the native polypeptide in the animal's genome by recombination, resulting in underexpression of the transporter. Microinjection adds genes to the genome, but does not remove them, and so is useful for producing an animal which expresses its own and added polypeptides, resulting in overexpression of the polypeptides.

In one means available for producing a transgenic animal, with a mouse as an example, is as follows: Female mice are mated, and the resulting fertilized eggs are dissected out of their oviducts. The eggs are stored in an appropriate medium such as M2 medium. DNA or cDNA encoding a polypeptide of this invention is purified from a vector by methods well known in the art. Inducible promoters may be fused with the coding region of the DNA to provide an experimental means to regulate expression of the trans-gene. Alternatively, or in addition, tissue specific regulatory elements may be fused with the coding region to permit tissue-specific expression of the transgene. The DNA, in an appropriately buffered solution, is put into a microinjection needle and the egg to be injected is put in a depression slide. The needle is inserted into the pronucleus of the egg, and the DNA solution is injected. The injected egg is then transferred into the oviduct of a pseudopregnant mouse (a mouse stimulated by the appropriate hormones to maintain pregnancy but which is not actually pregnant), where it proceeds to the uterus, implants, and develops to term. As noted above, microinjection is not the only method for inserting DNA into the egg cell, and is used here only for exemplary purposes.

This invention provides for a process for identifying a chemical compound which specifically binds to a mammalian GIR receptor which comprises contacting cells containing DNA encoding and expressing on their cell surface the mammalian GIR receptor, wherein such cells do not normally express the mammalian GIR receptor, with the compound under conditions suitable for binding, and detecting specific binding of the chemical compound to the mammalian GIR receptor. This invention also provides a process for identifying a chemical compound which specifically binds to a mammalian GIR receptor which comprises contacting a membrane fragment from a cell extract of cells containing DNA encoding and expressing on their cell surface the mammalian GIR receptor, wherein such cells do not normally express the mammalian GIR receptor, with the compound under conditions suitable for binding, and detecting specific binding of the chemical compound to the mammalian GIR receptor. In an embodiment, the mammalian GIR receptor is a human GIR receptor. In a further embodiment, the mammalian GIR receptor has substantially the same amino acid sequence as shown in SEQ ID NO: 2, SEQ ID NO:4, or SEQ ID NO:6. In another embodiment, the mammalian GIR receptor has the identical amino acid sequence as shown in SEQ ID NO: 2, SEQ ID NO:4, or SEQ ID NO:6. In another embodiment, the compound is a compound not previously known to bind to a mammalian GIR receptor. This invention further provides a compound determined by the above-described process.

This invention provides a method involving competitive binding for identifying a chemical compound that specifically binds to a mammalian GIR receptor comprising separately contacting cells expressing on their cell surface the mammalian GIR receptor, wherein such cells do not normally express the mammalian GIR receptor, with both the chemical compound and a second chemical compound known to bind to the receptor, and with only the second chemical compound, under conditions suitable for binding of both compounds, and detecting specific binding of the chemical compound to the mammalian GIR receptor, a decrease in the binding of the second chemical compound to the mammalian GIR receptor in the presence of the chemical compound indicating that the chemical compound binds to the mammalian GIR receptor.

This invention also provides a process involving competitive binding for identifying a chemical compound which specifically binds to a mammalian GIR receptor which comprises separately contacting a membrane fraction from a cell extract of cells expressing on their cell surface the mammalian GIR receptor, wherein such cells do not normally express the mammalian GIR receptor, with both the chemical compound and a second chemical compound known to bind to the receptor, and with only the second chemical compound, under conditions suitable for binding of both compounds, and detecting specific binding of the chemical compound to the mammalian GIR receptor, a decrease in the binding of the second chemical compound to the mammalian GIR receptor in the presence of the chemical compound indicating that the chemical compound binds to the mammalian GIR receptor.

This invention provides a method of screening a plurality of chemical compounds not known to bind to a mammalian GIR receptor to identify a compound which specifically binds to the mammalian GIR receptor, which comprises (a) contacting cells transfected with and expressing DNA encoding the mammalian GIR receptor with a compound known to bind specifically to the mammalian GIR receptor; (b) contacting the preparation of step (a) with the plurality of compounds not known to bind specifically to the mammalian GIR receptor, under conditions permitting binding of compounds known to bind the mammalian GIR receptor; (c) determining whether the binding of the compound known to bind to the mammalian GIR receptor is reduced in the presence of the compounds within the plurality of compounds, relative to the binding of the compound in the absence of the plurality of compounds; and if so (d) separately determining the binding to the mammalian GIR receptor of compounds included in the plurality of compounds, so as to thereby identify the compound which specifically binds to the mammalian GIR receptor.

This invention provides for a method of screening a plurality of chemical compounds not known to bind to a mammalian GIR receptor to identify a compound which specifically binds to the mammalian GIR receptor, which comprises (a) preparing a cell extract from cells transfected with and expressing DNA encoding the mammalian GIR receptor, isolating a membrane fraction from the cell extract, contacting the membrane fraction with a compound known to bind specifically to the mammalian GIR receptor; (b) contacting the preparation of step (a) with the plurality of compounds not known to bind specifically to the mammalian GIR receptor, under conditions permitting binding of compounds known to bind the mammalian GIR receptor; (c) determining whether the binding of the compound known to bind to the mammalian GIR receptor is reduced in the presence of the compounds within the plurality of compounds, relative to the binding of the compound in the absence of the plurality of compounds; and if so (d) separately determining the binding to the mammalian GIR receptor of compounds included in the plurality of compounds, so as to thereby identify the compound which specifically binds to the mammalian GIR receptor.

This invention provides a method of detecting expression of a mammalian GIR receptor by detecting the presence of mRNA coding for the mammalian GIR receptor which comprises obtaining total mRNA from the cell and contacting the mRNA so obtained from the nucleic acid probe under hybridizing conditions, detecting the presence of mRNA hybridizing to the probe, and thereby detecting the expression of the mammalian GIR receptor by the cell.

This invention provides for a method of detecting the presence of a mammalian GIR receptor on the surface of a cell which comprises contacting the cell with the antibody under conditions permitting binding of the antibody to the receptor, detecting the presence of the antibody bound to the cell, and thereby detecting the presence of a mammalian GIR receptor on the surface of the cell.

This invention provides a method of determining the physiological effects of varying levels of activity of mammalian GIR receptors which comprises producing a transgenic, nonhuman mammal whose levels of mammalian GIR receptor activity are varied by use of an inducible promoter that regulates mammalian GIR receptor expression.

This invention provides a method of determining the physiological effects of varying levels of activity of mammalian GIR receptors which comprises producing a panel of transgenic, nonhuman mammals each expressing a different amount of mammalian GIR receptor.

This invention provides a method for identifying an antagonist capable of alleviating an ailment wherein the ailment is alleviated by decreasing the activity of a mammalian GIR receptor comprising administering a compound to the transgenic, nonhuman mammal and determining whether the compound alleviates the physical and behavioral abnormalities displayed by the transgenic, nonhuman mammal as a result of overactivity of a mammalian GIR receptor, the alleviation of the ailment identifying the compound as an antagonist. This invention provides an antagonist identified by the above-described method. This invention further provides a pharmaceutical composition comprising an antagonist identified by the above-described method and a pharmaceutically acceptable carrier. This invention further provides a method of treating an ailment in a subject wherein the ailment is alleviated by decreasing the activity of a mammalian GIR receptor which comprises administering to the subject an effective amount of the pharmaceutical composition, thereby treating the ailment.

This invention provides a method for identifying an agonist capable of alleviating an ailment in a subject wherein the ailment is alleviated by increasing the activity of a mammalian GIR receptor comprising administering a compound to the transgenic, nonhuman mammal, and determining whether the compound alleviates the physical and behavioral abnormalities displayed by the transgenic, nonhuman mammal, the alleviation of the ailment identifying the compound as an agonist. This invention also provides an agonist identified by the above-described method. This invention further provides a pharmaceutical composition comprising an agonist identified by the above-described method and a pharmaceutically acceptable carrier. This invention further provides a method of treating an ailment in a subject wherein the ailment is alleviated by increasing the activity of a mammalian GIR receptor which comprises administering to the subject an effective amount of the pharmaceutical composition, thereby treating the ailment.

This invention provides for a method for diagnosing a predisposition to a disorder associated with the activity of a specific mammalian allele which comprises: (a) obtaining DNA of subjects suffering from the disorder; (b) performing a restriction digest of the DNA with a panel of restriction enzymes; (c) electrophoretically separating the resulting DNA fragments on a sizing gel; (d) contacting the resulting gel with a nucleic acid probe capable of specifically hybridizing with a unique sequence included within the sequence of a nucleic acid molecule encoding a mammalian GIR receptor and labeled with a detectable marker; (e) detecting labeled bands which have hybridized to the DNA encoding a mammalian GIR receptor labeled with a detectable marker to create a unique band pattern specific to the DNA of subjects suffering from the disorder; (f) preparing DNA obtained for diagnosis by steps (a)-(e); and (g) comparing the unique band pattern specific to the DNA of subjects suffering from the disorder from step (e) and the DNA obtained for diagnosis from step (f) to determine whether the patterns are the same or different and to diagnose thereby predisposition to the disorder if the patterns are the same. In an embodiment, the disorder associated with the activity of a specific mammalian allele is diagnosed.

This invention provides a method of preparing the purified mammalian GIR receptor which comprises: (a) inducing cells to express the mammalian GIR receptor; (b) recovering the mammalian GIR receptor from the induced cells; and (c) purifying the mammalian GIR receptor so recovered.

This invention provides a method of preparing the purified mammalian GIR receptor which comprises: (a) inserting nucleic acid encoding the mammalian GIR receptor in a suitable vector; (b) introducing the resulting vector in a suitable host cell; (c) placing the resulting cell in suitable condition permitting the production of the isolated mammalian GIR receptor; (d) recovering the mammalian GIR receptor produced by the resulting cell; and (e) purifying the mammalian GIR receptor so recovered.

This invention also provides a process for determining whether a chemical compound is a mammalian GIR receptor antagonist which comprises contacting cells transfected with and expressing DNA encoding the mammalian GIR receptor with the compound in the presence of a known mammalian GIR receptor agonist, under conditions permitting the activation of the mammalian GIR receptor, and detecting a decrease in mammalian GIR receptor activity, so as to thereby determine whether the compound is a mammalian GIR receptor antagonist. In an embodiment, the mammalian GIR receptor is a human GIR receptor.

This invention provides for a pharmaceutical composition comprising an amount of a mammalian GIR receptor antagonist determined by the above-described process effective to reduce activity of a mammalian GIR receptor and a pharmaceutically acceptable carrier. In a further embodiment, the mammalian GIR receptor antagonist is not previously known.

This invention provides a process for determining whether a chemical compound specifically binds to and inhibits activation of a mammalian GIR receptor, which comprises separately contacting cells producing a second messenger response and expressing on their cell surface the mammalian GIR receptor, wherein such cells do not normally express the mammalian GIR receptor, with both the chemical compound and a second chemical compound known to activate the mammalian GIR receptor, and with only the second chemical compound, under conditions suitable for activation of the mammalian GIR receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the mammalian GIR receptor. In a farther embodiment, the second messenger response comprises chloride channel activation and the change in second messenger response is a smaller increase in the level of inward chloride current ill the presence of both the chemical compound and the second chemical compound than in the presence of only the second chemical compound.

In an embodiment, the mammalian GIR receptor is a human GIR receptor. In another embodiment, the human GIR receptor has substantially the same amino acid sequence as that shown in SEQ ED NO: 2, SEQ ID NO:4, or SEQ ID NO:6. In another embodiment, the human GIR receptor has an amino acid sequence, identical to the amino acid sequence shown in SEQ ID NO: 2, SEQ ID NO:4, or SEQ ID NO:6.

This invention provides for a pharmaceutical composition comprising an amount of a mammalian GIR receptor antagonist determined by the above-described process effective to reduce activity of a mammalian GIR receptor and a pharmaceutically acceptable carrier. In a further embodiment, the mammalian GIR receptor antagonist is not previously known.

This invention provides a method of screening a plurality of chemical compounds not known to inhibit the activation of a mammalian GIR receptor to identify a compound which inhibits the activation of the mammalian GIR receptor, which comprises: (a) contacting cells transfected with and expressing the mammalian GIR receptor with the plurality of compounds in the presence of a known mammalian GIR receptor agonist, under conditions permitting activation of the mammalian GIR receptor; (b) determining whether the activation of the mammalian GIR receptor is reduced in the presence of the plurality of compounds, relative to the activation of the mammalian GIR receptor in the absence of the plurality of compounds; and if so (c) separately determining the inhibition of activation of the mammalian GIR receptor for each compound included in the plurality of compounds, so as to thereby identify the compound which inhibits the activation of the mammalian GIR receptor. In an embodiment, the mammalian GIR receptor is a human GIR receptor.

This invention also provides a pharmaceutical composition comprising a compound identified by the above-described method effective to decrease mammalian GIR receptor activity and a pharmaceutically acceptable carrier. This invention also provides a pharmaceutical composition comprising a compound identified by the above-described methods effective to decrease mammalian GIR receptor activity and a pharmaceutically acceptable carrier.

In one embodiment, the ailment is a stress-related defect in which the antagonist would decrease the expression of GIR in the nervous system. In another embodiment, the behavior sensitization is sensitization initiated by repetitive stress administration. In another embodiment, the behavior is sensitization initiated by repetitive drug administration. The present invention provides a method of treating an ailment in a subject wherein the ailment is alleviated by decreasing the activity of a mammalian GIR receptor comprising administering to the subject an amount of compound which is a mammalian GIR receptor antagonist effective to treat the ailment. This invention provides a method of treating an ailment in a subject wherein the ailment is alleviated by decreasing the activity of a mammalian GIR receptor comprising administering to the subject an amount of compound which is a mammalian GIR receptor antagonist effective to treat the ailment.

In a further embodiment, the ailment is an impulsive addiction or compulsive disorder caused by the neural adaptation or behavioral sensitization of the GIR receptor. In yet another embodiment, the ailment is a drug addiction in which the antagonist preferably reduces or prevents the GIR protein activation.

This invention will be better understood from the examples which follow. However, one skilled in the art will readily appreciate that the specific methods and results discussed are merely illustrative of the invention as described more fully in the claims which follow thereafter.

### EXAMPLES

Materials and Methods

Animals. Male adult Sprague-Dawley rats (200-250 g, Harlan Sprague-Dawley Laboratories, Indianapolis, Ind.) were housed for a minimum of 2 weeks in a vivarium prior to use in experiments. Animals were sacrificed by decapitation and brains were quickly removed and placed on ice for dissection. Dissection was performed using a coronal rodent brain matrix as described (Segal, Kuczenski, 1974). The most anterior brain region was used for assays of prefrontal cortex after removing the olfactory bulb. The caudate was taken as a circular punch 3 mm in diameter. Nucleus accumbens was taken as the portion of this same slice ventral to an extension of the anterior commissure. After removing the hippocampus, substantia nigra and ventral tegmentum were taken as a single section ventral to the medial lemniscus and medial to the dentate gylus. Following dissection, brain regions were frozen in liquid nitrogen and stored at −70 (C prior to use.

Drugs. D-Amphetamine (Research Biochemicals, Natick, Mass.) was dissolved in 0.9% saline. Drug concentrations were calculated as the free base. All injections were subcutaneous in a final volume of 1 ml/kg body weight.

RNA Preparation and Reverse Transcription. Total RNA was isolated from rat prefrontal cortex by single-step guanidinium isothiocyanate/acid phenol extraction using the TRI Reagent (Molecular Research Center, Cincinnati, Ohio) according to the manufacturer's instruction. The purified RNA was used for the rat GIR cDNA cloning, amplification of both 5′ and 3′ cDNA ends, and RT-PCR Southern blot analyses. The concentrations of RNA samples were determined by spectrophotometric measurements at 260 and 280 nm. First-strand cDNA was synthesized from total RNA using a random hexanier primer (Promega, Madison, Wis.) as described previously (Miao et al., 2000).

PCR. A 166 bp rat cDNA fragment was isolated from medial prefrontal cortex by differential display (N. Richtand, unpublished data). Using the BLASTN program on National Center for Biotechnology Information (NCBI) web server, we found that this fragment shared 81% identity to the mouse GIR. Then we designed two oligonucleotides (RGIR20 and RGIR21) in the 166 bp stretch and one oligonucleotide (MGIR1) in transmembrane domain I of mouse GIR which is outside the 166 bp rat-mouse matched region. The oligonucleotides were: RGIR20 (5′-AGCAGAGGCTGGTTCAGTCA-3′) (SEQ ID NO:7), RGIR21 (5′-GTACCTATCTCGGTTCCAGT-3′) (SEQ ID NO:8), and MGIR1 (5′-CTCTTCGGTAATGTCCTGGTC-3′, 256-276 bp, Genbank M80481) (SEQ ID NO:9). Amplification was conducted with primers MGIR1 and RGIR21 in a thermal cycler for 30 cycles of 94° C./30 sec, 50° C./30 sec, 68° C./2 min using Platinum Taq polymerase (Life Technologies, Bethesda, Md.). One-third of the RT-PCR product was subjected to Southern blot analysis using the nested GIR-specific primer RGIR20 as probe. The blotted membrane was washed in 2×SSC at 60° C. and exposed to Kodak X-Omat AR film at −80° C. The 1.3 kb PCR product, amplified from the prefrontal cortex and identified by Southern blot analysis, was purified by agarose gel electrophoresis and subcloned into pGEM-T vector (Promega, Madison, Wis.). Positive clones were selected by colony-hybridization using the RGIR20 probe again, and insert sequences were determined by DNA sequencing. Sequence analysis demonstrated a 98% sequence identity between the rat and mouse GIR cDNA (Harrigan et al., 1991).

5′-RACE and 3′-RACE. After obtaining the partial rat GIR cDNA, 5′- and 3′-RACE (rapid amplification of cDNA ends) were performed to determine the putative leading exon and 3′ noncoding region of rat GIR using the SMARTTM RACE cDNA Amplification Kit (Clontech, Palo Alto, Calif.). Total RNA from rat prefrontal cortex was used as template to synthesize the first strand cDNAs for 5′- and 3′-end according to the manufacturer's instruction. The first strand 5′-RACE-Ready cDNA was synthesized with 5′-RACE cDNA Synthesis Primer (5′-CDS) and SMART II Oligonucleotide, and the first strand 3′-RACE-Ready cDNA was synthesized with 3′-RACE cDNA Synthesis Primer (3′-CDS) (Clontech, Palo Alto, Calif.).

The GIR-specific primers were designed from the obtained partial sequence of rat GIR, RGSP1 (5′-TTATAACAAGTGCTGCTCATGGCAAACCAGTGG-3′, 1301-1333 bp in FIG. 1) (SEQ ID NO:10); RGNSP1 (5′-CAAAGTAGAGGGCGTTGTTGGTGTGG-3′, 1267-1296 bp in FIG. 1) (SEQ ID NO:11); RGSP2 (5′-CCTCTTCGCTCTCTGCTGGTTCCCC-3′, 1209-1233 bp in FIG. 1) (SEQ ID NO:12); and RGNSP2 (5′-CAACTGCTATGTCCTCCTTCTGTCCAGC-3′, 1236-1262 bp in FIG. 1) (SEQ ID NO:13). Primary 5′-RACE amplification was performed in 50 μl final volume using 2.5 μl 1/100 diluted 5′-RACE-ready cDNA template, 2 μM of GIR-specific RGSP1 and Universal Primer Mix (UPM), and Advantage 2 Polymerase Mix (Clontech, Palo Alto, Calif.). The 3′-RACE was performed similarly by using 3′-RACE-ready cDNA and GIR-specific RGSP2. PCR amplification was conducted in a thermal cycler for initial 5 cycles of 94° C./5 sec, 72° C./3 min; followed by 5 cycles of 94° C./5 sec, 70° C./10 sec, 72° C./3 min; and ended with 35 cycles of 94° C./5 sec, 68° C./10 sec, 72° C./3 min. The RACE amplification products were confirmed by Southern blot analysis with nested GIR-specific RNGSP1 for 5′-RACE product, and RNGSP2 for 3′-RACE product, respectively. The resulting hybridized DNA fragments were gel-purified and subcloned into pGEM-T vector (Promega, Madison, Wis.). Positive clones were selected and insert sequences were determined by DNA sequencing.

Direct DNA Sequencing. Plasmid DNA was prepared using a Qiagen DNA purification kit (Qiagen, Valencia, Calif.). Double-stranded DNA was directly sequenced at the University of Cincinnati DNA Core Facility, and confirmed by complementary strand sequence.

Tissue Distribution. Tissue-specific expression of GIR was investigated by Northern blot analysis and reverse transcription-PCR (RT-PCR) Southern blot analysis. A rat multiple-tissue Northern blot of poly(A)+RNA from heart, brain, spleen, lung, liver, skeletal muscle, kidney, and testis was purchased from Clontech (Palo Alto, Calif.). The rat GIR-specific cDNA probe (1209-1575 bp in FIG. 1) was labeled by random priming with [α-32P]dCTP, and probed the membrane at 68° C. for 1 hr in ExpressHyb Solution (Clontech, Palo Alto, Calif.). Each blot was rinsed with 2×SSC containing 0.05% SDS at room temperature for 40 min, washed twice with 0.1×SSC containing 0.1% SDS at 50° C. for 40 min, and then autoradiographed using Kodak X-Omat AR film at −80 ° C. Finally, each blot was stripped and rehybridized with a rat c-actin cDNA probe (Clontech, Palo Alto, Calif.) as an internal standard.

Various brain regions from male Sprague-Dawley rats, including caudate putamen, cerebellum, hippocampus, hypothalamus, nucleus accumbens, olfactory tube, prefrontal cortex, substantia nigra/ ventral tegmentum area, were dissected. Total RNA was isolated from these brain regions and immortalized rat amygdalar neuronal cells (kindly provided by Dr. John W. Kasckow, University of Cincinnati) (Mulchahey et al., 1999). The GIR-specific primers RGSP2: 5′-CCTCTTCGCTCTCTGCTGGTTCCCC-3′ (SEQ ID NO:12); and P2: 5′-CGGCCACAGTGGGTTCCAC-3′ (SEQ ID NO:14), which amplify nucleotides 1209-1575 (367 bp), were used for PCR amplification in a thermal cycler for 30 cycles of 94° C./30 sec, 60° C./30 sec, 72° C./2 min. The RT-PCR products were then subjected to Southern blot analysis with a nested GIR-specific probe (RGNSP2: 5′-CAACTGCTATGTCCTCCTTCTGTCCAGC-3′, 1236-1262 bp in FIG. 1), (SEQ ID NO:13). The blotted membrane was washed 4 times in 2×SSC at 60° C. and exposed to Kodak X-Omat AR film at −80° C. An aliquot of the first-strand cDNA was amplified for rat beta-actin mRNA as an internal control with beta-actin-specific primers (5′-GAACCCTAAGGCCAACCGTG-3′, SEQ ID NO:15; and 5′-TGGCATAGAGGTCTTTACGG-3′, SEQ ID NO:16), for 25 cycles of 94° C./30 sec, 60° C./30 sec, 72° C./1 min, and hybridized with a nested beta-actin-specific probe (5′-CGCACGATTTCCCTCTCAGC-3′, SEQ ID NO:17).

In Situ Hybridization. Assessment of GIR mRNA expression was accomplished using probes that recognize all GIR mRNA forms (367 bp, complementary to the 1209-1575bp region of rat GIR mRNA, FIG. 1). Labeling reactions included 60 μCi 35S-UTP (specific activity 1800 Ci/mmol), 1× transcription buffer, 15 mM dithiothreitol, 200 μM GTP, CTP and ATP, 10 μM UTP, 40 U placental RNase inhibitor, 1 μg linearized plasmid DNA, and 20 U of appropriate RNA polymerase (T3 or T7, Roche Molecular Biochemicals, Indianapolis, Ind.). Reactions were incubated at 37 (C for 90 min, the DNA template was then removed by RNase-free DNase I digestion for 15 min at 37 (C, and reaction mix was diluted to 100 μl with diethylpyrocarbonate (DEPC)-treated water and ethanol precipitated with 7.5 M ammonium acetate.

Brain sections were fixed for 10 min in 4% buffered paraformaldehyde, and rinsed twice in 5 mM DEPC-treated potassium PBS (pH 7.5) (UPBS) (5 min), twice in KPBS/0.2% glycine, and twice in KPBS. Sections were then acetylated by a 10 min treatment with 0.25% acetic anhydride and 0.1 M triethanolamine (pH 8.0), rinsed twice in 0.2×SSC (5 mm) and dehydrated in a graded ethanol series. Labeled probes were added to a hybridization buffer containing 50% formamide, 20 mM Tris-HCl pH 7.5, 1 mM EDTA, 335 mM NaCl, 1× Denhardt's, 200 μg/ml salmon sperm DNA, 150 μg/ml yeast transfer RNA, 20 mM dithiothreitol, and 10% dextran sulfate. Probes were denatured for 15 min at 65 (C and 50 μl (1×106 cpm) of diluted probe applied to each slide. Slides were coverslipped, placed in moistened chambers, and incubated overnight at 55 (C. Following hybridization, coverslips were removed in 0.2×SSC and rinsed in fresh 0.2×SSC for 10 min. Sections were treated with RNase A (50 ug/ml) for 30 min at 37 (C and transferred to fresh 2×SSC, then rinsed three times in 0.2×SSC (10 min per wash) followed by a 1 h wash in 0.2×SSC at 65 (C. Sections were dehydrated in a graded ethanol series, dried at room temperature and exposed for 14-21 days to Kodak BioMAX film (Eastman Kodak Co, Rochester, N.Y.). Hybridization controls included 1) sections hybridized with sense-strand probe generated from the same vector construct, and 2) sections preincubated in RNase A (50 μg/ml, 30 min, 37 (C) prior to hybridization with antisense probe.

GIR mRNA Expression Following Chronic Amphetamine Administration. Male Sprague-Dawley rats (200-250g, Harlan Sprague-Dawley Laboratories, Indianapolis, Ind.) received subcutaneous injection of either d-amphetamine (AMPH) (2.5 mg/kg) or saline (1 ml/kg) in their home cages once daily for 5 consecutive days (days 1-5). On day 9, four days following completion of this pre-treatment regimen, all animals were transferred to Residential Activity Chambers (RACs) (Segal and Kuczenski, 1987; Richtand et al., 2000), and received saline injection (1 ml/kg) on days 10 and 11 to measure conditioned response to injection. On day 12, seven days following completion of the pre-treatment regimen, all rats received AMPH (2.5 mg/kg) and locomotor activity was determined by photobeam monitoring for 150 minutes following injection. Locomotor activity is expressed as crossovers, defined as the number of times the animal crosses into any of four quadrants subdividing the enclosure. All animals were sacrificed by decapitation 4 hours following injection and total RNA was extracted from prefrontal cortex for determination of GIR mRNA expression. In a separate experiment, rats were injected with either AMPH (2.5 mg/kg) or saline (1 ml/kg) for 5 consecutive days, and sacrificed by decapitation 7 days after the last injection without the final AMPH challenge. Thus, four groups were generated: SAL-AMPH (Saline×5 days and AMPH at challenge), AMPH-AMPH (AMPH×5 days and AMPH at challenge), SAL (Saline×5 days), and AMPH (AMPH×5 days).

Construction of Competitor DNA and Quantitative RT-PCR. To construct the mutant template for competitive PCR, a 20 bp GIR sequence (5′-TCGCTCTCTGCTGGTTCCCC-3′, 1213-1232 bp in FIG. 1; SEQ ID NO: 18) was incorporated into the upstream primer so that a rat GIR cDNA fragment was amplified by RT-PCR using the chimeric upstream primer (5′-TCGCTCTCTGCTGGTTCCCCTCCCTTCATCTACTGCTGGC-3′, 1213-1232 bp in FIG. 1; SEQ ID NO:19) and the downstream primer (5′-CGGCCACAGTGGGTTCCAC-3′, 1575-1557 in FIG. 1; SEQ ID NO:13). The 262 bp PCR product was subcloned into pGEM-T vector (Promega, Madison, Wis.). The plasmid DNA was purified (Qiagen, Valencia, Calif.) and insert was verified by DNA sequencing. A fragment containing the mutant rat GIR template (262 bp) was released by EcoRI digestion and purified by agarose gel electrophoresis. DNA concentration was determined spectrophotometrically. Serial dilutions of mutant template were prepared.

The amount of GIR mRNA was quantitated by competitive RT-PCR as described previously (Miao et al., 2000). Briefly, reverse transcription was performed with 2 μg of total RNA using a non-specific random hexamer primer (Promega, Madsion, Wis.). One μl of first strand cDNA was mixed with known amounts of linearized competitor DNA ranging from 0.5 μg to 16 μg, and subjected to co-amplification with GIR-specific primers (5′-TCGCTCTCTGCTGGTTCCCC-3′, 1213-1232 bp, SEQ ID NO:IS; and 5′-CGGCCACAGTGGGTTCCAC-3′, 1575-1557 bp in FIG. 1, SEQ ID NO:14). PCR was run for 30 cycles of 94° C./1 min, 60° C./2 min 72° C./3 min, and the PCR products were electrophoresed on 1.2% agarose gel. The intensities of bands, corresponding to the products from the GIR mRNA and the competitor with 101-bp size difference, was analyzed densitometrically by BioImage system combined with NIH software (Bio-Rad, Hercules, Calif.). The intensity ratios between the two bands were logarithmically plotted against the known initial input of competitor template, and the amount of GIR mRNA in the total RNA samples was calculated by linear regression. Control PCR with water or RNA template gave no amplification product (data not shown).

Statistical Analysis. Group data are expressed as mean±SE. Statistically analysis was performed by unpaired Student's t-test. Differences were considered significant at P<0.05.

Results

Identification and Characterization of Rat GIR cDNA

We have cloned and characterized the full-length GIR cDNA from rat prefrontal cortex by RT-PCR, 5′-RACE and 3′-RACE. Complete sequencing of the cDNA reveals a 1266-bp open reading frame flanked by a 314-bp 5′-untranslated region and a 2100-bp 3′-untranslated region (FIG. 1). A polyadenylation signal is present at position 3622, 16 nt upstream of the poly(A) tail. A potential translation initiation site (ATG) is assigned to the methionine codon at nucleotide positions 315-317 because it matches the vertebrate consensus sequences at +4 position and is predicted to initiate translation of GIR, though it shows a suboptimal sequence at the −3 position (Kozak, 1991).

Amino Acid Sequence and Its Comparison with Other Receptors

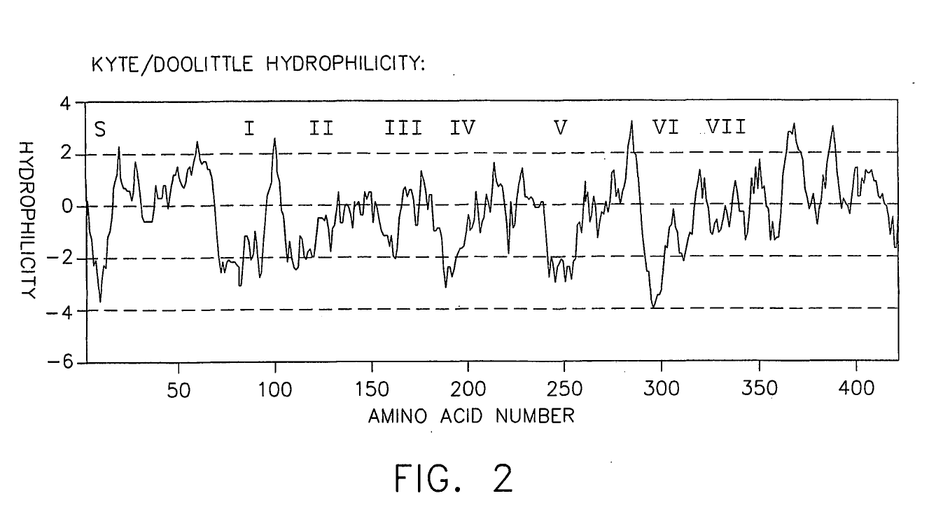

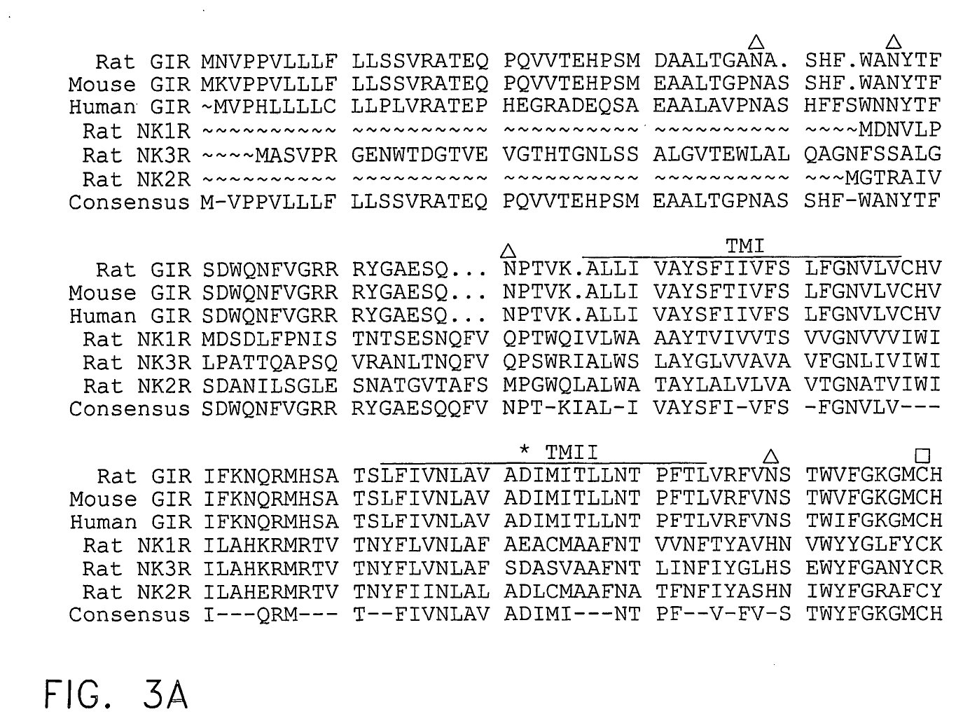

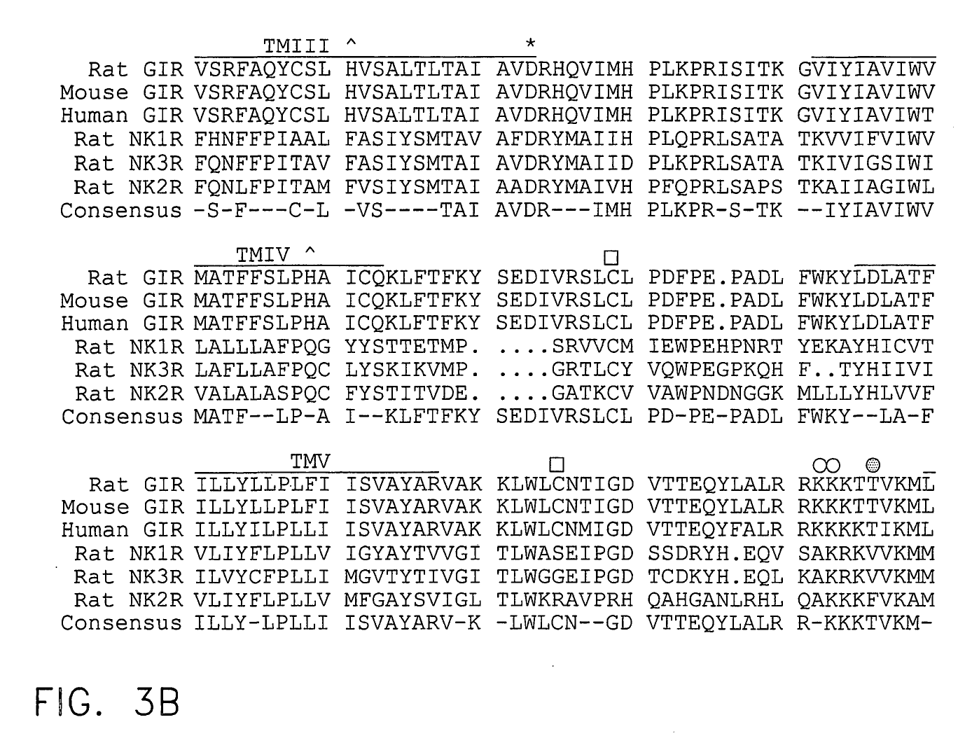

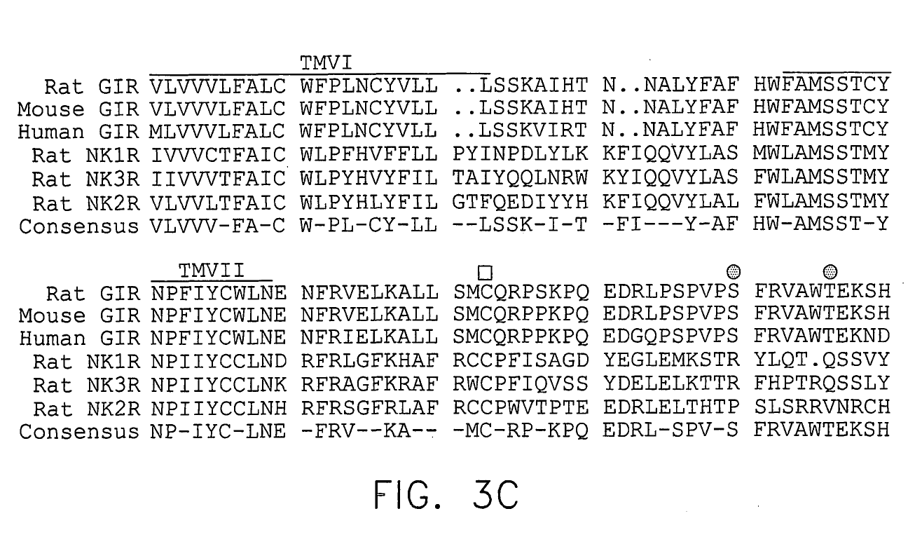

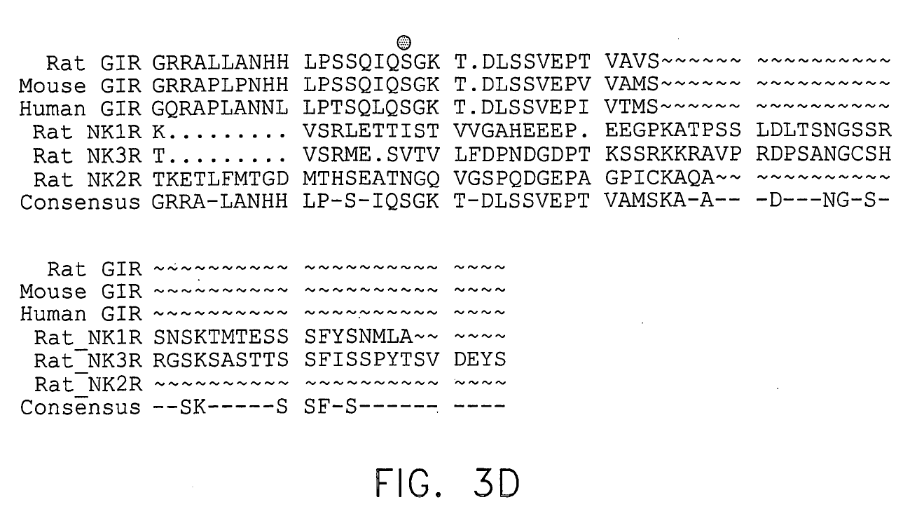

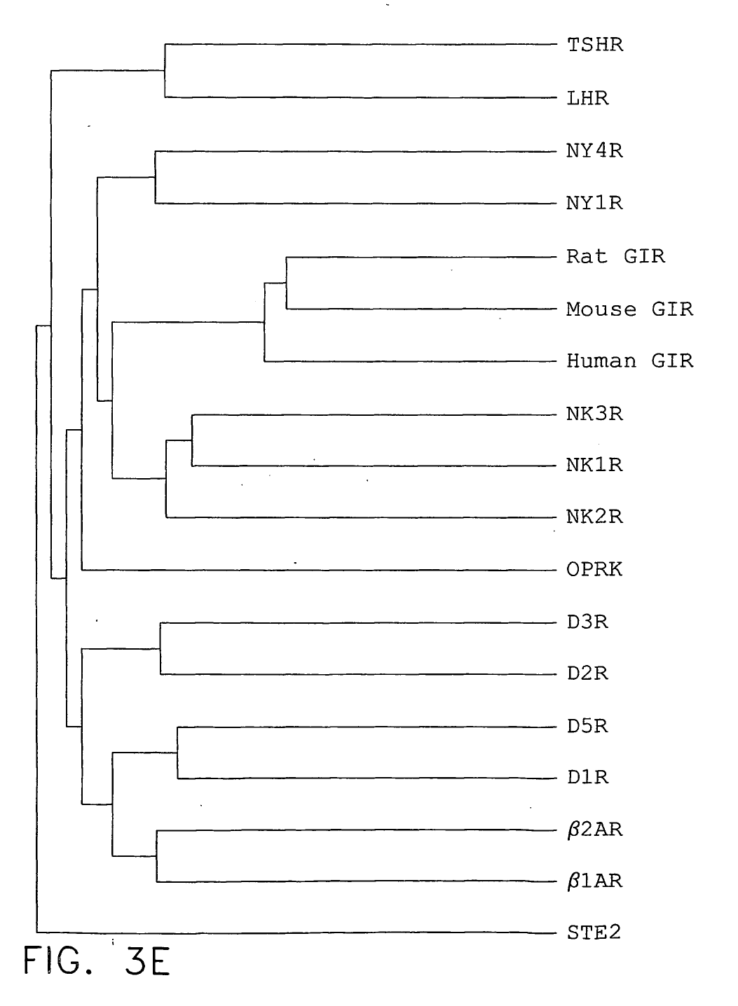

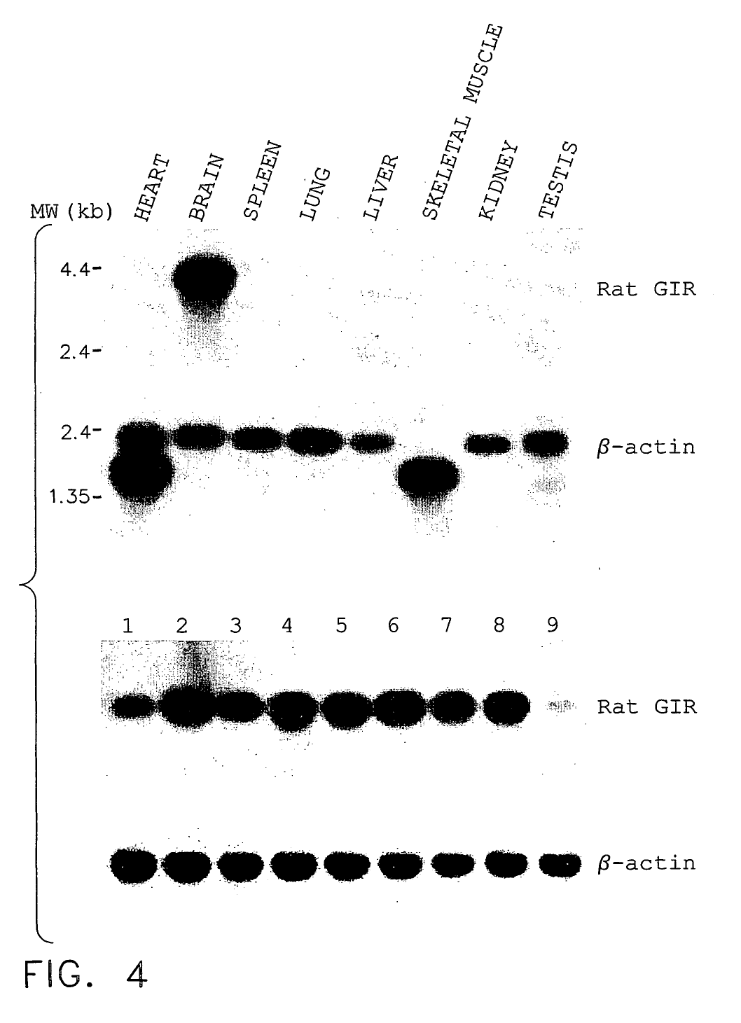

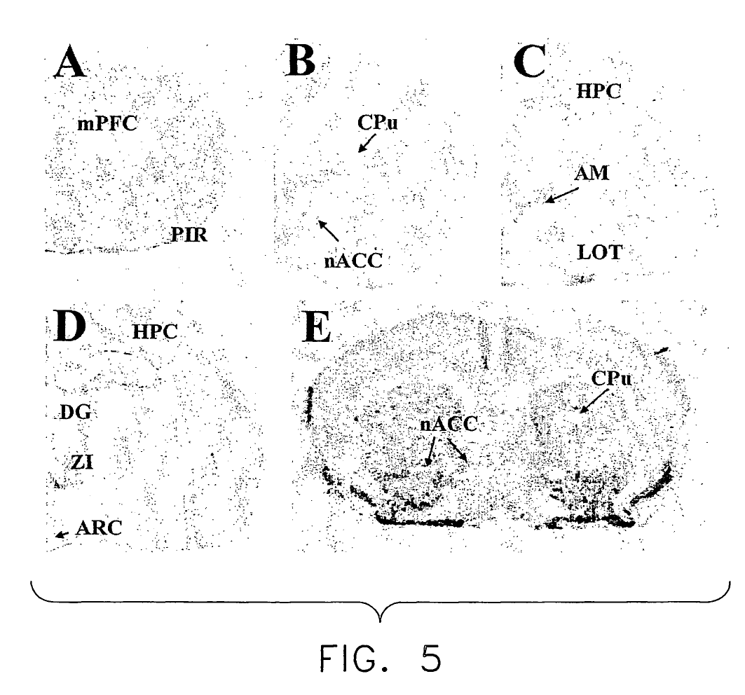

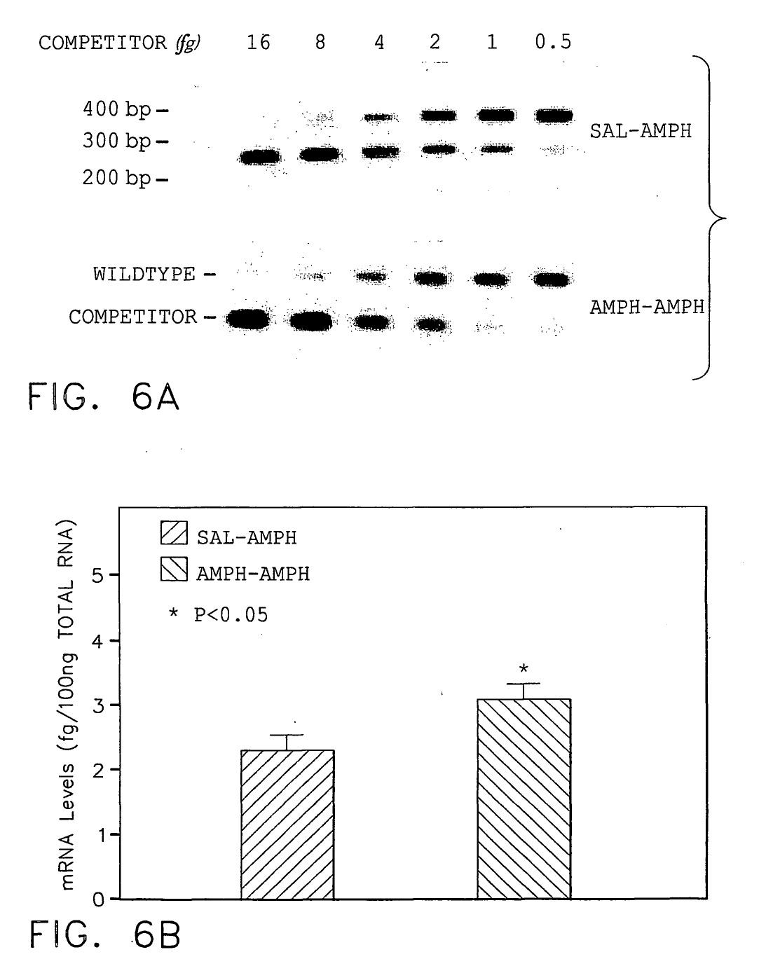

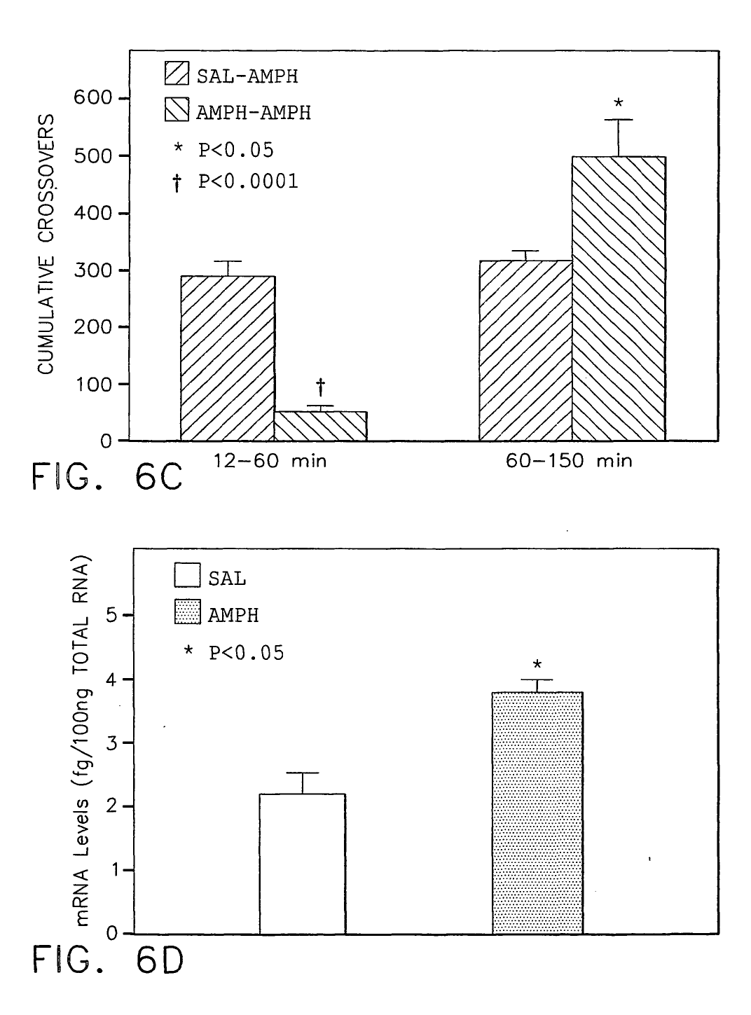

Based on amino acid sequence derived from the rat GIR cDNA, this gene encodes a protein of 422 amino acids with a relative molecular mass of 48 kDa. The hydrophobicity plot of the protein sequence reveals the presence of 7 distributed hydrophobic regions throughout the polypeptide, indicating that GIR belongs to the G-protein-coupled receptor superfamily (FIG. 2). The 7 putative transmembrane x-helices of GIR consist of a continuous stretch of 17-23 uncharged amino-acid residues, except that transmembrane domains II, III, and IV contain Asp114 116, His155, Asp167 and His203, respectively. FIG. 2 shows the hydrophobic N-terminal region with the presence of a potential signal sequence (17-residue in length) and potential signal peptidase cleavage sites (von Heijne, 1986) located at Val15 and Ala17.

The primary amino-acid sequences of rat GIR were compared in the Genbanlc database, and reveal a significant similarity to other G-protein-coupled receptors (FIG. 3A). Overall, the rat GIR shares highest sequence identity with mouse (97%) and human GIR (88%), limited but close identity with rat substance-P receptor (NK-1, 33.7%), neurokinin A receptor (NK-2, 31.3%), and neurokinin B receptor (NK-3, 34.3%). The rat GIR shows sequence identity to the transmembrane domains I (50%), II (38%), III (27%), IV (33%), V (36%), VI (43%) and VII (70%) of substance-P receptor, with an overall sequence identity of 42.4%. It also shows significant sequence identity to the 1st (33%), 2nd (53%), and 3rd intracellular loops (31%), with more limited sequence identity to the N-terminus (22%), 1st (25%), 2nd (27%) and 3rd extracellular loops (8%) as well as C-tenninus (20%) of substance-P receptor. FIG. 3A shows the alignment of these receptors illustrating several conserved regions in their amino acid sequences. The rat GIR has several structural characteristics common to the membranes of the G-protein-coupled receptor superfamily. Four potential N-linlced glycosylation sites are identified as Asn38, Asn45, Asn 66 and Asn133 according to the consensus sequence of Asn-X-Ser/Tir. Three of them are located in the N-terminus whereas one is in the 1st extracellular loop. Several consensus sites for possible phosphorylation by protein kinase C and protein kinlase A exist in the 3rd intracellular loop and C-tenninus, respectively. Several cysteine residues are identified in the intracellular and extracellular loops as well as C-terminus, respectively. In comparison with the mouse GIR, the rat GIR has a deletion involving one amino acid residue at position 40. A dendrogram was constructed using the Wisconsin Package Version 10, Genetics Computer Group (GCG, Madison, Wis.) and the clusters of GIR and tachykinin receptors are closely related as compared to the other members in the G-protein-coupled receptor superfamily (FIG. 3B).

Tissue Distribution of Rat GIR

The tissue distribution of GIR expression in rats was examined by Northern blot analysis and RT-PCR Southern blot analysis. A major mRNA species was detected in brain by Northern blot analysis using rat GIR cDNA as probe (FIG. 4A). The expression was not detected in peripheral tissues, including heart, spleen, lung, liver, skeletal muscle, kidney and testis (FIG. 4A). RT-PCR Southern blot analysis identified GIR mRNA expression in transformed rat amygdalar cells as well as various brain regions including caudate putamen, cerebellum, hippocampus, hypothalamus, nucleus accumbens, olfactory tube, prefrontal cortex, and to a lesser degree in substantia nigra/ventral teginental area (VTA) (FIG. 4B).

Cellular Localization of GIR mRNA in Rat Forebrain

In situ hybridization analysis was performed to assess GIR mRNA expression in rat forebrain (FIG. 5A, B, C). Hybridizations revealed high levels of GIR expression in the nucleus of the lateral olfactory tract, dorsolateral septum, tenia tecta, nucleus reunions of the thalamus, zona incerta, hippocampal pyramidal cells and the dentate gyrus (FIG. 5A-D). Significant hybridization signal was also observed in all regions of neocortex and in limbic cortical regions, notably including infralimbic, prelimbic and anterior cingulate regions comprising the rodent prefrontal cortical system (FIG. 5A, B, C). Significant positive hybridization was also observed in the olfactory bulbs, posterior divisions of the bed nucleus of the stria terminalis, midline thalamic nuclei, the ventromedial and ventrobasal complexes of the thalamus (FIG. 5A-D). Low levels of GIR mRNA could be detected in neuroendocrine regulatory regions of the hypothalamus, including the medial preoptic area, paraventricular nucleus, supraoptic nucleus and suprachiasmatic nucleus. Low but definitely detectable levels of GIR mRNA were observed in the nucleus accunibens shell. In no case was positive hybridization observed in sections hybridized with sense-strand probes or sections pretreated with RNAse A.

Notably, ill situ hybridization did not detect GIR mRNA in striatum (FIG. 5B), whereas GIR was detected in these regions by RT-PCR (FIG. 4B). A probe generated from the PCR fragment used to detect GIR in striatum yielded the same anatomical distribution as the original cRNA, indicating that the in situ hybridization and RT-PCR target sequences were not alternatively spliced RNA species. Thus, detection of GIR mRNA in striatum is likely due to enhanced sensitivity of the RT-PCR technique. It remains to be determined whether the low levels of GIR mRNA present in the striatum is sufficient to generate meaningful levels of GIR protein.

It is important to note that the distribution of GIR mRNA in rat brain differed substantially from that seen in mouse brain (FIG. 5B, D). Mouse brain sections hybridized with rat GIR probe showed intense expression of GIR in the striatum and nucleus throughout the nucleus accumbens (FIG. 5E). In rat, GIR mRNA expression in the caudate-putamen did not exceed background hybridization levels (FIG. 5B), and nucleus accumbens expression was quite low and limited to the shell region.

Response of Rat GIR Expression to Chronic Amphetamine Administration

The effect of amphetamine on GIR expression was studied following chronic amphetamine administration. The expression levels of GIR mRNA in rat prefrontal cortex after behavioral sensitization to amphetamine were quantitatively determined by competitive RT-PCR. The representative competitive PCR image is shown in FIG. 6A. The GIR mRNA levels were calculated from the linear regression plot of the ratio plotted logarithmically against the initial input of competitor DNA, as shown in FIG. 6B. In the experiment with amphetamine administration for 5 days followed by a challenge injection 7 days after drug ceased, the GIR mRNA levels in prefrontal cortex of AMPH-AMPH rats were significantly increased by 1.38-fold compared to SAL-AMPH rats (2.97(±0.26 vs. 2.27(±0.22 fg/100 ng total RNA, n=6, P<0.05) (FIG. 6B). Animal behavior was also determined following this amphetamine treatment regimen. In agreement with earlier studies (Segal and Kuczensld, 1994), amphetamine pretreatment resulted in robust behavioral sensitization under these conditions, as evidenced by the significant decrease in locomotion at 12-60 min post-injection during the focused stereotypy phase of AMPH response (50.20±12.63 vs. 286.00±27.57 crossovers/12-60 min, n=6, P<0.0001) and increased locomotion at 60-150 min during the post-stereotypy locomotion phase of AMPH response (496.67±70.28 vs. 312.25±29.17 crossovers/60-150min, n=6, P<0.05) (FIG. 6C).

In a separate experiment with chronic amphetamine exposure but no challenge injection,, the expression levels of GIR were quantitatively determined by competitive RT-PCR 7 days following an identical 5-day pretreatment regimen with amphetamine or saline. A similar up-regulation of GIR mRNA was observed 7 days after amphetamine ceased. The GIR mRNA levels in prefrontal cortex of AMPH rats were significantly increased by 1.70-fold compared to SAL rats (3.65±0.22 vs. 2.15±0.33 fg/100 ng total RNA, n=6, P<0.05) (FIG. 6D).

1. A method of predicting a patient's likelihood of developing a stress reward disorder comprising:
(a) Obtaining DNA from a human subject; and
(b) Detecting in said DNA a human glucocorticoid-induced receptor (GIR) gene wherein the gene indicates a potential susceptibility to an addiction reward disorder.

2. The method of claim 1 wherein detecting a human GIR receptor gene comprises:
(a) Subjecting said DNA of the subject to digestion by restriction enzyme;
(b) Hybridizing the DNA to a labeled probe specifically binding to a portion of a GIR gene allele of the human GIR receptor; and
(c) Determining the presence of the allele of the human GIR receptor.

3. The method of claim 2 wherein the probe is a recombinant cDNA or a fragment thereof.

4. The method of claim 3 wherein the DNA is separated by electrophoresis according to size.

5. The method of claim 4 wherein the stress reward disorder is a disorder selected from the group consisting of polysubstance abuse, drug addiction, nicotine addiction, alcohol addiction and cocaine addiction.

6. The method of claim 5 wherein the detection step is by restriction fragment length polymorphism (RFLP) or PASA.

7. A method of detecting a genetic potential susceptibility to stress reward disorder in a human subject comprising:
(a) Obtaining DNA from the subject;
(b) Subjecting the DNA of the subject to digestion by a restriction enzyme; and
(c) Hybridizing the DNA to a labeled probe specifically binding to the nucleotide SEQ ID NO: 1, SEQ ID NO: 3 or SEQ ID NO: 5; and
(d) Determining the presence of the GIR receptor.

8. A method of detecting the presence of a stress reward disorder susceptibility locus in an individual comprising analyzing a sample of DNA from the individual for the presence of a DNA polymorphism of the GIR gene sequence wherein the polymorphism is associated with stress reward disorder.

9. A method of genetically diagnosing an addiction reward disorder in an individual comprising analyzing a DNA sample from an individual for the presence of a DNA polymorphism associated with addiction reward disorder wherein the DNA polymorphism is in the GIR gene region and the presence of the DNA polymorphism is an indication that the individual has addiction reward disorder.

10. A method of treating an individual phenotypically diagnosed with an addiction reward disorder comprising:
(a) Analyzing a DNA sample from an individual phenotypically diagnosed with an addiction reward disorder for the presence or absence of a DNA polymorphism associated with addiction reward disorder wherein the DNA polymorphism is located within a region of the GIR gene allele and wherein the presence of a DNA polymorphism associated with addiction reward disorder confirms the diagnosis; and
(b) Selecting a treatment plan that is most effective for individuals phenotypically diagnosed as having addiction reward disorder.

11. A method of diagnosis of GIR receptor sensitization in a human subject which comprises the steps of:
(a) Obtaining a DNA sample from a human subject;
(b) Amplifying the DNA sample and
(c) Analyzing the amplified DNA of step (a) to determine whether the sample comprises at least one disease causing sequence abnormality with respect to the human GIR nucleotide sequence as set forth in SEQ ID NO: 3 or SEQ ID NO: 5 or a sequence encoding the human GIR amino acid sequence as set forth in SEQ ID NO: 4 or SEQ ID NO: 6 such abnormality leading to a decrease or increase in the GIR activity and being indicative of a GIR disorder.

12. A method for identifying a chemical compound which specifically binds to a mammalian GIR receptor which comprises contacting cells transfected with DNA encoding and expressing on their cell surface the mammalian GIR receptor or a membrane fraction from such cells with the compound under conditions suitable for binding and detecting specific binding of the chemical compound to the receptor wherein such cells or membrane fraction do not normally express the receptor and wherein the receptor has an amino acid sequence substantially similar to the amino acid sequence of SEQ ID NO: 2, SEQ ID NO: 4 or SEQ ID NO: 6.

13. A method for determining whether a chemical compound is a mammalian GIR receptor agonist which comprises contacting cells transfected with and expressing DNA encoding a mammalian GIR receptor or a membrane fraction from such cells with the compound under conditions permitting the activation of the GIR receptor and detecting an increase in receptor activity so as to thereby determine whether the compound is a receptor agonist wherein the receptor has an amino acid sequence of substantially similar to the amino acid sequence of SEQ ID NO: 2, SEQ ID NO: 4 or SEQ ID NO: 6.

14. A method for determining whether a chemical compound is a mammalian GIR receptor antagonist which comprises contacting cells transfected with and expressing DNA encoding a mammalian GIR receptor or a membrane fraction from such cells with the compound under conditions permitting the activation of the GIR receptor and detecting an increase in receptor activity so as to thereby determine whether the compound is a receptor antagonist wherein the receptor has an amino acid sequence of substantially similar to the amino acid sequence of SEQ ID NO: 2, SEQ ID NO: 4 or SEQ ID NO: 6.

15. The method of claim 14 wherein the cells are mammalian cells.

16. The method of claim 15 wherein the mammalian cells are non-neuronal in origin.

17. A method for determining whether a chemical compound specifically binds to and activates a mammalian GIR receptor which comprise:
(a) Contacting cells transfected with DNA encoding and expressing on their cell surface the GIR receptor and producing a second messenger response upon activation of the receptor with the chemical compound under conditions suitable for activation of the receptor;
(b) Measuring the second messenger response in the presence and in the absence of the chemical compound; and
(c) Wherein a change in the second messenger response in the presence of the chemical compound indicates that the compound activates the receptor and wherein such cells do not normally express the GIR receptor;
(d) Wherein the receptor has an amino acid sequence substantially similar to the amino acid sequence of SEQ ID NO: 2, SEQ ID NO: 4 or SEQ ID NO: 6.

18. A method for determining whether a chemical compound specifically binds to and inactivates a mammalian GIR receptor which comprise:
(a) Contacting cells transfected with DNA encoding and expressing on their cell surface the GIR receptor and producing a second messenger response upon activation of the receptor with the chemical compound under conditions suitable for inactivation of the receptor;
(b) Measuring the second messenger response in the presence and in the absence of the chemical compound; and
(c) Wherein a change in the second messenger response in the presence of the chemical compound indicates that the compound inactivates the receptor and wherein such cells do not normally express the GIR receptor;
(d) Wherein the receptor has an amino acid sequence substantially similar to the amino acid sequence of SEQ ID NO: 2, SEQ ID NO: 4 or SEQ ID NO: 6.

19. The method of claim 18 wherein the second messenger response comprises intra-cellular calcium levels.

20. A method of screening a plurality of chemical compounds not known to bind a mammalian GIR receptor to identify a compound which specifically binds to the receptor which comprises:
(a) Contacting cells transfected with and expressing DNA encoding the GIR receptor or a membrane fraction from such cells with a compound known to bind specifically to the GIR receptor;
(b) Contacting or a membrane fraction identical to those contacted in step (a) with the plurality of compounds not known to bind specifically to the receptor under conditions permitting binding of compounds known to bind the receptor;
(c) Determining whether the binding of the compound known to bind the receptor is reduced in the presence of one or more of the compounds within the plurality of compounds relative to the binding of the compound in the absence of the plurality of compounds; and
(d) Separately determining the binding to the receptor of each compound included in the plurality of compounds so as to thereby identify the compound or compounds which specifically binds to the GIR receptor and wherein the mammalian GIR receptor has an amino acid sequence substantially similar to the amino acid sequence of SEQ ID NO: 2, SEQ ID NO: 4 or SEQ ID NO: 6.

21. A method of screening a plurality of chemical compounds to identify a compound which activates the GIR receptor which comprises:
(a) Contacting cells transfected with and expressing the GIR receptor with the plurality of compounds to activate the GIR receptor or a membrane fraction from such cells under conditions permitting activation of the receptor;
(b) Determining whether the activity of the receptor is increased in the presence of one or more of the compounds within the plurality of compounds and separately determining which compounds activate the GIR receptor so as to thereby identify the compound or compounds that activate the receptor.

22. A lit for use in genetically detecting potential susceptibility to stress reward disorders in a human subject, said kit comprising:
(a) A first container means including a restriction enzyme capable of cleaving a human GIR receptor gene; and
(b) A second container means including a hybridization probe for detecting a human GIR receptor gene allele whose presence indicates susceptibility to addiction reward disorder.

23. The kit of claim 22 wherein the restriction enzyme is Taq I.

24. A lit for use in genetically detecting potential susceptibility to stress reward disorders in a human subject said kit comprising:
(a) A first container means comprising PASA primers specifically binding GIR receptor alleles characterizing susceptibility to addiction reward disorder; and
(b) A second container means comprising ingredients for PCR amplification of specific GIR gene alleles.

25. The method of claim 2 wherein the mammalian GIR receptor gene is substantially homolgous to at least 14 contiguous nucleotides of the sequence of SEQ ID NO: 3, SEQ ID NO:5, or SEQ ID NO:7.

26. The method of claim 2 wherein the mammalian GIR receptor gene encodes a polypeptide having substantial homology over at least 13 contiguous amino acids of the sequence of SEQ ID NO: 2, SEQ ID NO:4, or SEQ ID NO:6.

27. The method of claim 2 wherein the mammalian GIR receptor gene has at least 98% homology over at least 14 contiguous nucleotides of the sequence of SEQ ID NO: 3, SEQ ID NO:5, or SEQ ID NO:7.

28. The method of claim 2 wherein the mammalian GIR receptor gene encodes a polypeptide having at least 99% homology over at least 13 contiguous amino acids of the sequence of SEQ ID NO: 2, SEQ ID NO:4, or SEQ ID NO:6.

29. The method of claim 2 wherein the labeled probe is one or more oligonucleotides selected from the group consisting of SEQ ID NO: 8, SEQ ID NO:9, SEQ ID NO: 10, SEQ ID NO:11, SEQ ID NO: 12, SEQ ID NO:13, SEQ ID NO: 14, SEQ ID NO:15,. SEQ ID NO: 16, SEQ ID NO:17, SEQ ID NO: IS, and SEQ ID NO:19.

30. The kit of claim 22 wherein the labeled probe is one or more oligonucleotides selected from the group consisitng of SEQ ID NO: 8, SEQ ID NO:9, SEQ ID NO: 10, SEQ ID NO:1 1, SEQ ID NO: 12, SEQ ID NO:13, SEQ ID NO: 14, SEQ ID NO:15, SEQ ID NO: 16, SEQ ID NO:17, SEQ ID NO: 18, and SEQ ID NO:19.
